SEQUENCE LISTING (42 sequences)

SEQ ID NO: 1 - DNA, 348 bp, Homo sapiens
gaggtgcagctgttggagtctgggggaggcttggtacagcctggggggtccctgcgtctctcctgtgcagcctccggattcacctttagcagctatgccatgagctgggtccgccaggctccagggaagggtctagagtgggtctcagctattagtggtagtggtggtagcacatactacgcagactccgtgaagggccggttcaccatctcccgtgacaattccaagaacacgctgtatctgcaaatgaacagcctgcgtgccgaggacaccgcggtatattactgtgcgaaaagttatggtgcttttgactactggggtcagggaaccctggtcaccgtctcgagc

SEQ ID NO: 2 - protein, 116 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 3 - DNA, 324 bp, Homo sapiens
gacatccagatgacccagtctccatcctccctgtctgcatctgtaggagaccgtgtcaccatcacttgccgggcaagtcagagcattagcagctatttaaattggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagagttacagtacccctaatacgttcggccaagggaccaaggtggaaatcaaacgg

SEQ ID NO: 4 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 5 - DNA, 324 bp, Homo sapiens
gacatccagatgacccagtctccatcctccctgtctgcatctgtaggagaccgtgtcaccatcacttgccgggcaagtcagagcattcgaacgggggtagtttggtaccagcagaaaccagggaaagcccctaagctcctgatctatagtgcatcccatttgcaaagtggggtcccatcacgtttcagtggcagtggatctgggacagatttcactctcaccatcagcagtctgcaacctgaagattttgctacgtactactgtcaacagatttttacgaggcctgtgacgttcggccaagggaccaaggtggaaatcaaacgg

SEQ ID NO: 6 - protein, 108 aa, Homo sapiens
DIQMTQSPSSLSASVGDRVTITCRASQSIRTGVVWYQQKPGKAPKLLIYSASHLQSGVPSRFSGSGSGTDFTLTISSLQPEDFATYYCQQIFTRPVTFGQGTKVEIKR

SEQ ID NO: 7 - DNA, 145 bp, artificial sequence
  Mutiple cloning site sequence of pDOM1 and pDOM2
gtgaaaaaattattattcgcaattcctttagttgttcctttctattctcacagtgcacaggattacaaggacgacgatgacaagtcgacacactgcaggaggcggccgcagaacaaaaactcatctcagaagaggatctgaattc

SEQ ID NO: 8 - protein, 37 aa, artificial sequence
  Multiple cloning site of pDOM1 and pDOM2
VKKLLFAIPLVVPFYSHSDYKDDDDKEQKLISEEDLN

SEQ ID NO: 9 - DNA, 54 bp, Bacteriophage M13
gtgaaaaaattattattcgcaattcctttagttgttcctttctattctcactcc

SEQ ID NO: 10 - protein, 18 aa, Bacteriophage M13
MKKLLFAIPLVVPFYSHS

SEQ ID NO: 11 - DNA, 54 bp, Bacteriophage fd; identical to SEQ ID NO: 9

SEQ ID NO: 12 - protein, 18 aa, Bacteriophage fd
MKKLLFAIPLVVPFYSHS

SEQ ID NO: 13 - DNA, 69 bp, Bacteriophage M13
atgaagaagagtctggtgctgaaagcgagtgtagcggtggcaacgctggtgccgatgctgagttttgcg

SEQ ID NO: 14 - protein, 23 aa, Bacteriophage M13
MKKSLVLKASVAVATLVPMLSFA

SEQ ID NO: 15 - protein, 10 aa, Homo sapiens
EQKLISEEDL

SEQ ID NO: 16 - DNA, 33 bp, Homo sapiens
gaacaaaaactcatctcagaagaggatctgaat

SEQ ID NO: 17 - protein, 9 aa, Bacteriophage T7
DYKDDDDKG

SEQ ID NO: 18 - DNA, 24 bp, Bacteriophage T7
gattacaaggacgacgatgacaag

SEQ ID NO: 19 - protein, 6 aa, artificial sequence
  Synthetic peptide
HHHHHH

SEQ ID NO: 20 - DNA, 18 bp, artificial sequence
  DNA sequence encoding His tag peptide
catcatcatcaccatcac

SEQ ID NO: 21 - protein, 9 aa, Influenza virus
YPYDVPDYA

SEQ ID NO: 22 - DNA, 27 bp, Influenza virus
tatccttatgatgttcctgattatgca

SEQ ID NO: 23 - protein, 11 aa, Vesicular stomatitis virus
YTDIEMNRLGK

SEQ ID NO: 24 - DNA, 33 bp, Vesicular stomatitis virus
tatacagacatagagatgaaccgacttggaaag

SEQ ID NO: 25 - protein, 14 aa, Simian virus 5
GKPIPNPLLGLDST

SEQ ID NO: 26 - DNA, 42 bp, Simian virus 5
ggtaagcctatccctaaccctctcctcggtctcgattctacg

SEQ ID NO: 27 - protein, 11 aa, herpes simplex virus
QPELAPEDPED

SEQ ID NO: 28 - DNA, 33 bp, herpes simplex virus 7
cagcccgagctggcccccgaggaccccgaggac

SEQ ID NO: 29 - DNA, 69 bp, Bacteriophage M13
atgaaaaagagcctggtacttaaggcgagtgttgcggtggcgacgctggtcccgatgctgagttttgcg

SEQ ID NO: 30 - protein, 23 aa, Bacteriophage M13
MKKSLVLKASVAVATLVPMLSFA

SEQ ID NO: 31 - DNA, 69 bp, Bacteriophage M13
atgaagaaaagtctggtactgaaggcgagtgtggcggtggccactctggttccaatgcttagtttcgcg

SEQ ID NO: 32 - protein, 23 aa, Bacteriophage M13
MKKSLVLKASVAVATLVPMLSFA

SEQ ID NO: 33 - DNA, 50 bp, artificial sequence
  Oligonucleotide containing multiple cloning site
tgcacaggattacaaggacgacgatgacaagtcgacacactgcaggaggc

SEQ ID NO: 34 - DNA, 50 bp, artificial sequence
  Oligonucleotide containing multiple cloning site
ggccgcctcctgcagtgtgtcgacttgtcatcgtcgtccttgtaatcctg

SEQ ID NO: 35 - DNA, 40 bp, artificial sequence
  primer
gtggtgtcgacagaggtgcagctgttggagtctgggggag

SEQ ID NO: 36 - DNA, 44 bp, artificial sequence
  primer
gagtcaactgcggccgcgctcgagacggtgaccagggttccctg

SEQ ID NO: 37 - DNA, 41 bp, artificial sequence
  primer
gtggtgtcgacagacatccagatgacccagtctccatcctc

SEQ ID NO: 38 - DNA, 45 bp, artificial sequence
  primer
gagtcaactgcggccgcccgtttgatttccaccttggtcccttgg

SEQ ID NO: 39 - DNA, 24 bp, artificial sequence
  primer
atggttgttgtcattgtcggcgca

SEQ ID NO: 40 - DNA, 65 bp, artificial sequence
  primer
cgccaagctttggagcctttttttttggagatttttaacatgaaaaaattattattcgcaattcc

SEQ ID NO: 41 - DNA, 40 bp, artificial sequence
  primer
gcgcgaattcttattaattcagatcctcttctgagatgag

SEQ ID NO: 42 - DNA, 24 bp, artificial sequence
  primer
atgaggttttgctaaacaactttc

(19) United States
(12) Patent Application Publication
(10) Pub. No.: US 2004/0202995 A1
(43) Pub. Date: Oct. 14, 2004

(54) Nucleic acids, proteins, and screening methods

(75) Inventors: Rudolf Maria Theodora de Wildt, Cambridge (GB); Laurent Jespers, Cambridge (GB); Ian Michael Tomlinson, Cambridge (GB)

Correspondence Address: PALMER & DODGE, LLP, KATHLEEN M. WILLIAMS, 111 HUNTINGTON AVENUE, BOSTON, MA, 02199, US

(73) Assignee: Domantis

(21) Appl. No.: 10/409,814

(22) Filed: Apr. 9, 2003

(51) Int. Cl. C12Q 1/70; C07K 14/005; C12N 7/00; C07H 21/04; C12N 15/86

(52) U.S. Cl. 435/5; 435/235.1; 435/69.1; 435/252.3; 435/456; 530/350; 536/23.72; 530/388.3

(57) ABSTRACT

Herein, immunoglobulin variable region polypeptides are fused at their N-terminus to a bacterial signal sequence. In one embodiment, the immunoglobulin variable region polypeptides are fused to a tag that that is placed in between a bacteriophage signal sequence and the N-terminus of the immunoglobulin variable region polypeptide. In another embodiment, the N-termini of single-domain antibodies (dAb) are fused to a bacterial signal sequence in the absence of a tag. In a further embodiment, the immunoglobulin variable region antibody fusion proteins are fused to a bacteriophage coat protein and expressed on the surface of bacteriophage providing an efficient way to select small antibody fragments that are labeled and that have high affinity to target ligand.

### BACKGROUND OF THE INVENTION

Antibodies are versatile immunological reagents used both as reporter molecules and diagnostic agents. Traditional monoclonal antibodies are divalent and are highly useful because of their specific and high-affinity binding to antigen. However, small antibody fragments are proving to have the same utility.

The advent of recombinant techniques has allowed for the generation of monovalent synthetic antibody fragments, such as single-chain antibodies (scFVs) and Fab fragments that lack a portion or all of the antibody constant domains normally found in an intact antibody.

For example, scFVs lack all antibody constant regions wherein the V_{H} domain is directly linked to a V_{L} domain by a designed polypeptide linker sequence, and Fab fragment antibodies contain the antibody constant regions C_{H1} and C but lack constant domains C_{H2} and C_{H3}. Both scFVs and Fab fragments have been successfully displayed on the surface of bacteriophage, which has allowed for the selection of monovalent fragment antibodies with antigen binding affinities as high as their divalent counterparts, WO 00/70023 (Dyax Corporation).

The developments in antibody engineering and bacteriophage display technology have also lead to the understanding that a single antibody domain, either an individual V_{L} domain or an individual V_{H} domain, can function as specific antigen binding domain. The use of single-domain antibodies (dAbs) are an attractive alternative to scFVs or Fabs because they are much smaller in size and they have affinities comparable to that seen with scFVs. The smaller size of antibody fragments is advantageous to applications requiring, e.g. tissue penetration or rapid blood clearance. In addition, bacteriophage antibody library construction is much simpler and more efficient when single-domain antibodies are used instead of Fabs or scFvs. For example, U.S. Pat. No. 5,702,892 (U.S.A Health & Human Services) and WO 01/18058 (Novopharm Biotech Inc.) disclose bacteriophage display libraries and selection methods for V_{H} domain binding-fragments.

There is a need in the art for methods for generating small antibody reagents that can be efficiently purified, easily detected, and that have a desirable affinity to antigen.

### SUMMARY OF THE INVENTION

The invention contemplates the generation of immunoglobulin variable region polypeptide fusion proteins that can be easily purified and screened for binding to target ligand.

One aspect of the invention relates to a nucleic acid molecule that encodes a signal peptide/tag/immunoglobulin variable region polypeptide fusion protein. The nucleic acid molecule comprises a first DNA sequence encoding the signal peptide of a bacteriophage protein linked at its 3′ end to a second DNA sequence encoding a tag wherein the second DNA sequence is linked at its 3′ end to a third DNA sequence encoding an immunoglobulin variable region polypeptide or an antigen binding fragment thereof.

The invention further provides an immunoglobulin variable region polypeptide fusion protein molecule comprising a first amino acid sequence comprising a signal peptide of a bacteriophage protein that is linked at it's C-terminus to the N-terminus of a second amino acid sequence comprising a tag, wherein the second amino acid sequence is linked at its C-terminus to a third amino acid sequence comprising an immunoglobulin variable region polypeptide.

In one embodiment, the signal peptide of the immunoglobulin variable region polypeptide fusion protein is a signal peptide of a bacteriophage coat protein.

In another embodiment, the tag of the immunoglobulin variable region polypeptide fusion protein is a molecule that binds to a protein ligand, such as an antibody, or peptide, or protein receptor.

In a further embodiment the tag of the immunoglobulin variable region polypeptide fusion protein does not bind a protein ligand, but rather binds to a non-protein ligand such as a metal-chelate resin, glycosides, hydrophobic compounds or small molecules.

In a still further embodiment the tag of the immunoglobulin variable region polypeptide fusion protein is a fluorescent tag, a luminescent tag, or a chromogenic tag. In one aspect the tag is Flag, His, Myc, HA, VSV, or V5.

In one embodiment, the immunoglobulin variable region polypeptide of the immunoglobulin variable region polypeptide fusion protein is a variable light chain (V_{L}) (e.g., Vλ or Vκ).

In one aspect, the variable region polypeptide comprises an antigen binding fragment of the light chain variable domain (V_{L}).

In another embodiment, the immunoglobulin variable region polypeptide of the immunoglobulin variable region polypeptide fusion protein comprises a heavy chain variable domain (V_{H}).

In one aspect, the variable region polypeptide comprises an antigen binding fragment of the variable heavy chain (V_{H}).

In one embodiment, the immunoglobulin variable region polypeptide of the immunoglobulin variable region polypeptide fusion protein comprises first and second light chain variable domains (e.g., V_{L}-V_{L}). Alternatively, the immunoglobulin variable region polypeptide of the immunoglobulin variable region polypeptide fusion protein may comprise 3, 4, 5, 6, or more light chain variable domains.

In another embodiment the immunoglobulin variable region polypeptide of the immunoglobulin variable region polypeptide fusion protein comprises first and second heavy chain variable domains (V_{H}-V_{H}). Alternatively, the immunoglobulin variable region polypeptide of the immunoglobulin variable region polypeptide fusion protein may comprise 3, 4, 5, 6, or more heavy chain variable domains.

In one aspect the first and second variable domains are linked by a peptide linker.

In another aspect, the peptide linker is a (Gly₄Ser)ₙ repeat where n=1-8, preferably 3, 4, 5, or 6.

In another embodiment, immunoglobulin variable region polypeptide of the immunoglobulin variable region polypeptide fusion protein comprises a constant domain (e.g., one or more of C_{H}1, C_{κ}, C_{λ}, C_{H}2, C_{H}3, e.g., (optional hinge)-C_{H}2-C_{H}3.

In a further embodiment, the signal peptide of the immunoglobulin variable region polypeptide fusion protein is the signal peptide from a bacteriophage protein pill.

In another embodiment, the signal peptide of the immunoglobulin variable region polypeptide fusion protein is the signal peptide for bacteriophage protein pVIII, pVII, or pIX. In a further embodiment, the signal peptide of the immunoglobulin variable region polypeptide fusion protein is a signal peptide having 90%, 95%, 98%, and up to 99% homology with the signal peptide of pVIII, pVII, pIX. Homology between a nucleic acid sequences may be determined by sequence alignment using, for example, Basic BLAST (e.g., Version 2.0, Altschul et al., 1997, Nucleic Acids Res. 25: 3389-3402) set with default parameters (descriptions default=500; alignments default=100; expect=10; filter=off; matrix=BLOSUM62). When two known sequences are to be aligned, the “Blast 2 Sequences” program can be used to align and determine homology (bl2seq; Tatusova & Madden, 1999, FEMS Microbiol. Lett. 174:247-250). The “Blast 2 Sequences” program, available through the NCBI website can be used with default alignment parameters. This program produces the alignment of two given sequences using the BLAST engine for local alignment. Default parameters (for use with the BLASTN program only) are as follows: Reward for a match: 1; Penalty for a mismatch: −2; Strand option Both strands; open gap penalty 5; extension gap penalty 2; gap x_dropoff 50; expect 10.0; word size 11; and Filter (checked).

In one embodiment, the signal peptide of the immunoglobulin variable region polypeptide fusion protein is encoded by a sequence found in the genome of bacteriophage fd.

In another embodiment, the signal peptide of the immunoglobulin variable region polypeptide fusion protein is encoded by a sequence found in the genome of bacteriophage f1, M13, or IKe.

In an additional embodiment, the nucleic acid that encodes the signal peptide/tag/immunoglobulin variable region polypeptide fusion protein is further linked to a bacteriophage coat protein. The DNA sequence that encodes a bacteriophage coat protein is linked to the 3′ end of the sequence that encodes the immunoglobulin variable region polypeptide.

In one aspect, the DNA sequence of the bacteriophage coat protein is found in the genome of bacteriophage fl, fd, M13, or IKe. In a preferred embodiment, the bacteriophage coat protein DNA sequence is found in the genome of bacteriophage fd.

In a further embodiment, a nucleic acid library is generated that comprises a plurality of nucleic acids that encode a signal peptide/tag/immunoglobulin variable region polypeptide fusion protein or a plurality of nucleic acid sequences that encode a signal peptide/tag/immunoglobulin variable region polypeptide/bacteriophage coat protein fusion protein.

The invention further provides for a method for selecting from a repertoire of polypeptides a population of immunoglobulin variable region polypeptides that bind to a target ligand. The method entails contacting the polypeptide library with a target ligand and selecting a population of polypeptides which bind to the target ligand.

In a preferred embodiment the immunoglobulin variable region polypeptides are displayed on the surface of bacteriophage.

In a further embodiment the nucleic acid or polypeptide libraries of the present invention are present in E. coli strain TB1.

The present invention provides a tagged polypeptide comprising an immunoglobulin variable region polypeptide, wherein said tagged polypeptide is produced by the cleavage of the signal peptide from an immunoglobulin variable region polypeptide fusion protein as described herein.

The invention further provides a nucleic acid vector comprising a DNA sequence encoding a signal peptide of a bacteriophage protein linked at its 3′ end to a second DNA sequence encoding a tag, which is in turn linked at its 3′ end to a third DNA sequence encoding an immunoglobulin variable region polypeptide.

In one embodiment, the nucleic acid vector further comprises a lacZ promoter.

In a further embodiment, the nucleic acid vector further comprises a bacteriophage geneIII promoter.

In one embodiment, the nucleic acid vector is pDOM1.

In a further embodiment, the nucleic acid vector is pDOM2.

### BRIEF DESCRIPTION OF THE DRAWINGS

FIG. 1 shows the vector map of pDOM1.

FIG. 2 shows the vector map of pDOM2.

FIG. 3 shows the multiple cloning site of both pDOM1 and pDOM2 (nucleic acid sequence: SEQ ID NO: 7; amino acid sequence: SEQ ID NO: 8).

FIG. 4 shows the nucleic acid and amino acid sequences of V_{H} dummy (SEQ ID NO: 1 and 2, respectively), V_{L} dummy (SEQ ID NO: 3 and 4 respectively), and V_{L} BSA28 (SEQ ID NO: 5 and 6 respectively).

FIG. 5 shows a schematic representation of the vectors used in construction of V_{H} and V_{L} libraries.

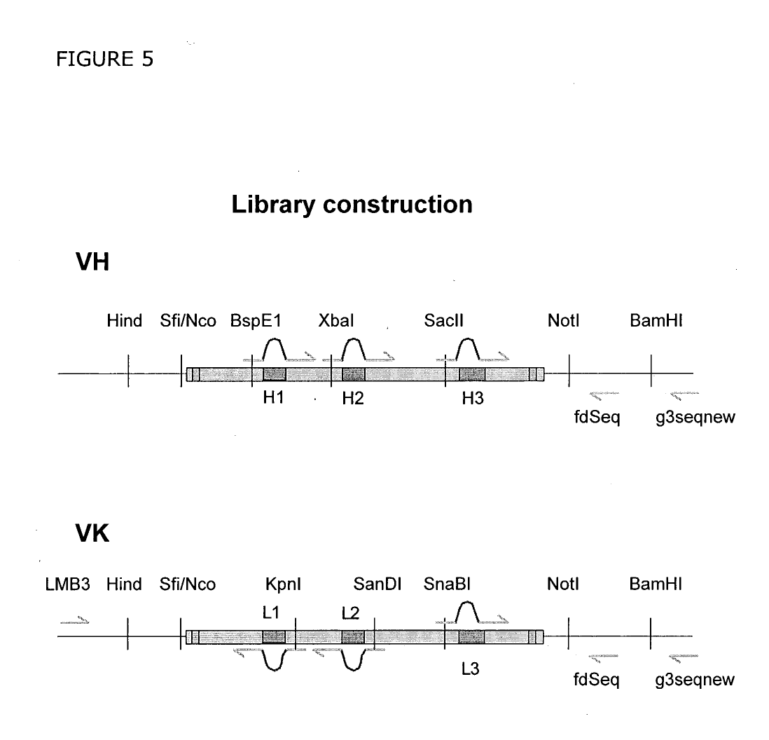

### DETAILED DESCRIPTION

The present invention relates to the generation of immunoglobulin variable region polypeptide fusion proteins and to immunoglobulin variable region polypeptide fusion protein libraries that can screened for binding to target ligand.

Definitions

As used herein, the term “isolated” with respect to nucleic acids, such as DNA, refers to molecules separated from other DNAs (i.e., separated form DNAs having a different nucleotide sequence). The term isolated, as used herein, also refers to a nucleic acid or peptide that is substantially free of cellular material (e.g., at least 95%, 98%, 99%, and up to 100% by weight), viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. The term “isolated” is also used herein to refer to polypeptides that are isolated from other cellular proteins and is meant to encompass both purified and recombinant polypeptides. Herein, an “isolated molecule” refers to either an isolated nucleic acid or an isolated polypeptide.

As used herein, a “signal peptide” or “leader” is a protein sequence that directs a polypeptide chain to which it is linked to the periplasm of bacteria, and the cleavage of which accompanies translocation of the polypeptide chain into the periplasm. A “signal peptide” or “leader” is a protein sequence that is encoded by a sequence found in the genome of a bacteriophage or a functional mutant or variant thereof. Non-limiting examples of signal peptides include the N-terminal signal peptide from the bacteriophage proteins pIII and pVIII, pVII, and pIX. Signal peptides of the present invention can be derived from a variety of bacteriophages such as, filamentous bacteriophage, lambda, T4, MS2, and the like. Filamentous bacteriophages include M13, Fd, or Ke. The bacteriophage signal peptide of the present invention can be a hybrid signal peptide that comprises amino acid sequences derived from at least 2 different signal peptide sequences, wherein at least one of the amino acid sequences is derived from a bacteriophage signal peptide.

As used herein, a “tag” refers to a polypeptide sequence (7, 8, 10, 15, 20, 25, and up to 30 amino acids) in length. A tag may possess a specific binding affinity for a peptide, protein ligand, or a non-peptide ligand, which permits the immunoglobulin variable region polypeptide to which it is fused to be either detected or isolated.

By “isolated” is meant that the immunoglobulin variable region polypeptide is separated from other cellular materials, some non-limiting methods of isolation include isolation of a single-domain antibody that has a poly-Histidine tag using a metal-chelate column, immunoprecipitation or affinity column purification using anti-tag antibodies.

By “detected” is meant a manner of determining the presence or absence of the tag, such as “detection” by western blot with anti-tag monoclonal antibody, by immunofluorescence, or the tag itself fluoresces. Non-limiting examples of suitable tags according to the invention include c-Myc, Flag, HA, and VSV-G, HSV, FLAG, V5, and HIS.

As used herein, the term “immunoglobulin variable region polypeptide fusion protein” refers to a fusion protein comprising a bacteriophage secretion signal sequence linked to a tag sequence, which is in turn linked to an immunoglobulin variable region polypeptide.

As used herein, the term “immunoglobulin variable region polypeptide” includes i) an antibody heavy chain variable domain (V_{H}), or antigen binding fragment thereof, with or without constant region domains ii) an antibody light chain variable domain (V_{L}), or antigen binding fragment thereof, with or without constant region domains iii) a V_{H} or V_{L} domain polypeptide without constant region domains linked to another variable domain (a V_{H} or V_{L} domain polypeptide) that is with or without constant region domains, (e.g., V_{H}-V_{H}, V_{H}-V_{L}, or V_{L}-V_{L}), and iv) single-chain Fv antibodies (scFv), that is a V_{L} domain polypeptide without constant regions linked to another V_{H} domain polypeptide without constant regions (V_{H}-V_{L}), the variable domains together forming an antigen binding site. In one embodiment of option (i), (ii), or (iii), each variable domain forms and antigen binding site independently of any other variable domain. Option (i) or (ii) can be used to form a Fab fragment antibody or an Fv antibody. Thus, as used herein, the term “immunoglobulin variable region polypeptide” refers to antibodies that may or may not contain constant region domains. In addition, as used herein, the term “immunoglobulin variable region polypeptide” refers to antigen binding antibody fragments that can contain either all or just a portion of the corresponding heavy or light chain constant regions. In addition, an “immunoglobulin variable region polypeptide”, as used herein includes light chain, heavy chain, heavy and light chains (e.g., scFv), Fd (i.e., V_{H}-C_{H}1) or V_{L}-C_{L}.

As used herein, the term “single-domain antibody” is synonymous with “dAb” and refers to an immunoglobulin variable region polypeptide wherein antigen binding is effected by a single variable region domain. A “single-domain antibody” as used herein, includes i) an antibody comprising heavy chain variable domain (V_{H}), or antigen binding fragment thereof, which forms an antigen binding site independently of any other variable domain, ii) an antibody comprising a light chain variable domain (V_{L}), or antigen binding fragment thereof, which forms an antigen binding site independently of any other variable domain, iii) an antibody comprising a V_{H} domain polypeptide linked to another V_{H} or a V_{L} domain polypeptide (e.g., V_{H}-V_{H} or V_{Hx-V}_{L}), wherein each V domain forms an antigen binding site independently of any other variable domain, and iv) an antibody comprising V_{L} domain polypeptide linked to another V_{L} domain polypeptide (V_{L}-V_{L}), wherein each V domain forms an antigen binding site independently of any other variable domain. As used herein, the V_{L} domain refers to both the kappa and lambda forms of the light chains.

As used herein, the term “linked” refers to peptide linkers, as well as to chemical bond linkages, such as linkages by disulfide bonds or by chemical bridges.

As used herein, “fragment thereof” (e.g., “antigen binding fragment thereof”) refers to an antigen binding region, i.e., portion of the whole immunoglobulin variable region polypeptide, wherein the portion has specificity for an antigen. For example, a fragment of an immunoglobulin variable region polypeptide can i) contain one or more constant region domain (e.g., C_{H}2-C_{H}3), ii) can consist of only variable region amino acid sequences without the amino acid sequence of the constant regions, or iii) it can consist of only a portion of the amino acid sequence of the variable region (i.e., comprising at least (e.g., only) those CDR and FW sequences necessary for antigen binding).

As used herein, “linked at its 3′ end” refers to a DNA sequence that is linked at its 3′ terminal end to another DNA sequence such that the linked nucleotide sequence encodes a fusion protein. The linkage can be direct, i.e. no intervening sequence, or indirect, that is mediated by a linker sequence wherein a linker sequence can consist of 3 to 120 nucleotides.

As used herein, the term “linked to the N terminus” refers to the fusion of a polypeptide sequence to the carboxyl terminus of another polypeptide. The fusion can be direct, i.e. no intervening sequence, or indirect, that is mediated by a short (e.g., about 2-40 amino acids) linker peptide.

As used herein, the term “linked at its C-terminus” refers to the fusion of the C-terminal end of a polypeptide sequence to the amino-terminus of another polypeptide. The fusion can be direct or may be mediated by a linker peptide (e.g., about 2-40 amino acids).

As used herein, the term “linker sequence” refers to a DNA sequence of about 3 to 120 (e.g., 3-60) nucleotides that encodes a “linker peptide”. A “linker peptide” is a short (e.g., about 1-40, e.g., 1-20 amino acids) sequence of amino acids that is not part of the sequence of either of two polypeptides being joined. A linker peptide is attached on its amino-terminal end to one polypeptide or polypeptide domain and on its carboxyl-terminal end to another polypeptide or polypeptide domain. Examples of useful linker peptides include, but are not limited to, glycine polymers ((G)ₙ) including glycine-serine and glycine-alanine polymers (e.g., a (Gly₄Ser)ₙ repeat where n=1-8, preferably, n=3, 4, 5, or 6).

As used herein, the term “directly linked” refers to the linkage of a bacteriophage signal peptide with an immunoglobulin variable region polypeptide, or to the linkage of an immunoglobulin variable region polypeptide with a bacteriophage coat protein, wherein the said peptides are fused in-frame in the absence of a linker peptide.

As used herein, “bacteriophage coat protein” refers to the bacteriophage proteins that provide the structure of bacteriophage particles. Non-limiting examples of bacteriophage coat proteins include, without limitation, M13 gene III, gene VIII; rd minor coat protein pill (Saggio et al., Gene 152:35, 1995); lambda D protein (Sternberg & Hoess, Proc. Natl. Acad. Sci. USA 92:1609, 1995; Mikawa et al., J. Mol. Biol. 262:21, 1996); lambda phage tail protein pV (Maruyama et al., Proc. Natl. Acad. Sci. USA 91:8273, 1994; U.S. Pat. No. 5,627,024); fr coat protein (WO96/11947; DD 292928; DD 286817; DD 300652); Φ29 tail protein gp9 (Lee, Virol. 69:5018, 1995); MS2 coat protein; T4 small outer capsid protein (Ren et al., Protein Sci. 5:1833, 1996), T4 nonessential capsid scaffold protein IPIII (Hong and Black, Virology 194:481, 1993), or T4 lengthened fibritin protein gene (Efimov, Virus Genes 10:173, 1995); PRD-1 gene III; Qβ3 capsid protein (as long as dimerization is not interfered with); and P22 tailspike protein (Carbonell and Villayerde, Gene 176:225, 1996).

As used herein, the term “phage vector” refers to a nucleic acid vector that comprises a phage packaging signal and a gene encoding at least one phage coat protein which allows for the incorporation of the nucleic acid into a phage particle.

As used herein, the term “phagemid” refers to a phage whose genome contains a plasmid that can be excised by co-infection of the host with a helper phage.

As used herein, the term “antibody” refers to an immunoglobulin molecule, or fragment thereof, that is capable of binding antigen. The term “antibody” is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques. Thus, the term includes segments of proteolytically-cleaved or recombinantly-prepared portions of an antibody molecule that are capable of selectively reacting with a specific protein. Non limiting examples of such proteolytic and/or recombinant fragments include Fab, F(ab′)₂, Fab′, Fv, dAbs (e.g., provided as V_{H} or V_{L} alone, V_{H}-V_{H}, or V_{L}-V_{L}) and single chain antibodies (scFv) containing a V_{L} and V_{H} domain joined by a peptide linker. The scFv's may be covalently or non-covalently linked to form antibodies having two or more binding sites. Thus, antibodies include polyclonal, monoclonal, or other purified preparations of antibodies and recombinant antibodies.

As used herein “repertoire” is a plurality of diverse variants, for example nucleic acid variants, that differ in nucleotide sequence, or to polypeptide variants that differ in amino acid sequence. According to the present invention, a repertoire of immunoglobulin variable region polypeptides comprises a population of variable region polypeptides that possesses a binding site for a target ligand. Generally, a “repertoire” includes a large number of variants, sometimes as many as 10⁹, 10¹⁰, 10¹¹, 10¹², or more. Smaller repertoires may be constructed and are extremely useful, particularly if they have been pre-selected to remove unwanted members, such as those including stop codons, incapable of correct folding or which are otherwise inactive. Such smaller repertories may comprise 10, 10², 10³, 10⁴, 10⁵, 10⁶ or more nucleic acids or polypeptides. Advantageously, smaller repertoires comprise between 10² and 10⁵ nucleic acids or polypeptides. According to the present invention, a repertoire of nucleotides encodes a corresponding repertoire of polypeptides.

As used herein, “library” refers to a mixture of heterogeneous polypeptides or nucleic acids containing in the range of 10¹² (e.g., 10⁹ to 10¹²) different members. Each member comprises one polypeptide or nucleic acid sequence variant of an immunoglobulin variable region. To this extent, library is synonymous with repertoire. Sequence differences between library members are responsible for the diversity present in the library. The library may take the form of a simple mixture of polypeptides or nucleic acids, or may be in the form organisms or cells, for example bacteria, viruses, animal or plant cells and the like, transformed with a library of nucleic acids. Preferably, each individual organism or cell contains only one member of the library. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

As used herein, a “target ligand” is the target molecule for which a specific binding member or members of the library repertoire are to be identified by virtue of the binding of the member(s) to the target ligand. A target molecule is a molecule for which an interaction with one or more members of the repertoire is sought. Thus, the term “target molecule” includes antigens, antibodies, enzymes, substrates for enzymes, lipids, any molecule expressed in or on any cell or cellular organism, any organic or inorganic small molecules, and any other molecules capable of interacting with a member of the polypeptide repertoire. The target ligands may themselves be antibodies.

As used herein a “subset” is a part of the repertoire. In the terms of the present invention, a subset of the repertoire can interact with the target molecule, and thus a subset of the repertoire can give rise to a detectable interaction on an array. For example, where the target molecule is a specific ligand for an antibody, a subset of antibodies capable of binding to the target ligand may be isolated. The subset may then be “varied” at particular residues, for example by mutagenesis, in order to modify the specific binding or affinity of target ligand interaction and further screened for specific, high affinity, target ligand antibody interactions.

As used herein, the term “specific binding” refers to the interaction of two molecules, e.g., an antibody and a protein or peptide, wherein the interaction is dependent upon the presence of particular structures on the respective molecules. For example, when the two molecules are protein molecules, a structure on the first molecule recognizes and binds to a structure on the second molecule, rather than to proteins in general. “Specific binding”, as the term is used herein, means that a molecule binds its specific binding partner with at least 2-fold greater affinity, and preferably at least 10-fold, 20-fold, 50-fold, 100-fold or higher affinity than it binds a non-specific molecule.

A. Vector Components & Construction

The present invention is based, in part, on the discovery that efficient, high level periplasmic secretion of immunoglobulin variable region polypeptides can be achieved in a prokaryote by fusing the N-terminus of an immunoglobulin variable region polypeptide to at the C-terminus of a bacteriophage secretion signal peptide. In particular, efficient, high level periplasmic expression of immunoglobulin variable region polypeptides is obtained by generating a DNA sequence that encodes a bacteriophage signal peptide/tag/immunoglobulin variable region polypeptide fusion protein, or by generating a DNA sequence that encodes a bacteriophage signal peptide/single-domain antibody fusion protein, and expressing the respective fusion proteins in a host, e.g., E. Coli.

To generate a bacteriophage signal peptide/tag/immunoglobulin variable region polypeptide fusion protein, the 5′ end of a DNA sequence encoding an immunoglobulin variable region polypeptide is linked to the 3′ end of a DNA sequence encoding a tag, wherein the tag is further linked at its' 5′ end to the 3′ end of a DNA sequence encoding a bacteriophage signal peptide. A DNA sequence that encodes a bacteriophage signal-peptide/single domain antibody fusion protein is generated by linking the 5′ end of a DNA sequence encoding a single-domain antibody to the 3′ end of a DNA sequence encoding a bacteriophage signal peptide. The linkage of the DNA sequences can be direct, i.e. no intervening sequence, or indirect, that is mediated by a linker sequence wherein a linker sequence can consist of 3 to 120 (e.g., 3-60) nucleotides. It is preferred that when a linker sequence is used, the linker sequence encodes a peptide including glycine-serine and/or glycine-alanine polymer.

Accordingly, the present invention provides a vector comprising a nucleic acid sequence that encodes a tag protein fused in frame in between a bacteriophage signal peptide and an immunoglobulin variable region polypeptide. The present invention also provides for a vector comprising a single-domain antibody that is fused to the C-terminus of a bacteriophage signal peptide in the absence of a tag. In one aspect of the invention, in order to incorporate the immunoglobulin variable region polypeptide into bacteriophage particles and to present the antibody fragment on the surface of bacteriophage, the immunoglobulin variable region polypeptide is further fused in frame to a bacteriophage coat protein.

1. Signal peptide Component

Herein, a signal peptide is fused in frame to the N-terminus of an immunoglobulin variable region polypeptide. The signal peptide of the present invention is a protein sequence that directs proteins to which it is fused to the periplasmic space of bacteria. In the present invention, the signal peptide is derived from a bacteriophage protein. Non-limiting examples of signal peptides useful in the present invention include the N-terminal signal peptide from the bacteriophage proteins pIII, pVIII, pVII, and pIX. The bacteriophage proteins can be from bacteriophages such as, filamentous bacteriophage, lambda, T4, MS2, and the like. In a preferred embodiment the signal peptide is derived from filamentous bacteriophage, such as M13, Fd, Fl, or Ke.

The DNA sequences encoding the signal peptides can be obtained from natural sources, for example amplified by PCR from bacteriophage genomic DNA, or can be made synthetically using synthetic oligonucleotides. Preferred signal peptide leader sequences, which may be used in the present invention include the following:

| M13 gene III signal/leader sequence (encodes pIII signal peptide) | |
|---|---|
| GTGAAAAAATTATTATTCGCAATTCCTTTAGTTGTTCCTTTCTATTCTCACTCC | (SEQ ID NO: 9) |
| MKKLLFAIPLVVPFYSHS | (SEQ ID NO: 10) |
| Fd gene III signal/leader sequence (encodes pIII signal peptide) | |
| GTGAAAAAATTATTATTCGCAATTCCTTTAGTTGTTCCTTTCTATTCTCACTCC | (SEQ ID NO: 11) |
| MKKLLFAIPLVVPFYSHS | (SEQ ID NO: 12) |
| gVIII signal/leader sequence (encodes gVIII signal peptide) | |
| ATGAAGAAGAGTCTGGTGCTGAAAGCGAGTGTAGCGGTGGCAACGCTGGTGCCGATGCTGAG | (SEQ ID NO: 13) |
| TTTTGCG | |
| MKKSLVLKASVAVATLVPMLSFA | (SEQ ID NO: 14) |
| gIX signal/leader sequence (encodes gIX signal peptide) | |
| ATGAAAAAGAGCCTGGTACTTAAGGCGAGTGTTGCGGTGGCGACGCTGGTCCCGATGCTGAG | (SEQ ID NO: 29) |
| TTTTGCG | |
| MKKSLVLKASVAVATLVPMLSFA | (SEQ ID NO: 30) |
| gVII signal/leader sequence (encodes gVII signal peptide) | |
| ATGAAGAAAAGTCTGGTACTGAAGGCGAGTGTGGCGGTGGCCACTCTGGTTCCAATGCTTAG | (SEQ ID NO: 31) |
| TTTCGCG | |
| MKKSLVLKASVAVATLVPMLSFA | (SEQ ID NO: 32) |

Additional signal sequences useful in the present invention are disclosed in WO03/004636.

Partial signal sequences and variants may also be used as long as the encoded signal peptide sequence directs the polypeptide sequence to which it is attached to the periplasm of bacteria. In one aspect of the invention, hybrid signal peptides that comprise amino acid sequences from at least 2 different signal peptides are used. In a preferred aspect, the hybrid signal sequence comprises an amino acid sequence from a signal peptide derived from a bacteriophage virus as well as an amino acid sequence from a signal peptide derived from a prokaryotic organism (e.g., bacterium).

2. Tag Component

Herein, an amino acid tag is fused in frame in between a bacteriophage signal peptide and an immunoglobulin variable region polypeptide. In a preferred embodiment, the tag of the present invention has a specific binding affinity for a peptide or protein ligand, or a non-peptide ligand, which allows the immunoglobulin variable region polypeptide to which it is fused to be either, detected or isolated. For example, the tag may comprise a unique epitope for which antibodies are readily available, or the tag can comprise metal-chelating amino acids.

In another embodiment, the tag comprises an amino acid that is labeled with a detectable marker. Detectable markers include, for example, radioisotopes, fluorescent molecules, chromogenic molecules, luminescent molecules, and enzymes. Useful detectable markers in the present invention include biotin for staining with labeled streptavidin conjugate, fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold. Patents teaching the use of such detectable markers include U.S. Pat. Nos. 3,817,837; 3,850,752; 3,939,350; 3,996,345; 4,277,437; 4,275,149; and 4,366,241, the entireties of which are incorporated by reference herein.

Non-limiting examples of suitable tags according to the invention include c-Myc, Flag, HA, and VSV-G, HSV, FLAG, V5, and HIS. Sequences for tags useful in the present invention are shown in Table 1.

TABLE 1

| | Amino Acid | Nucleic Acid | |
|---|---|---|---|
| Tag Name | Sequence | Sequence | Reference/Origin |
| c-myc | EQKLISEEDL | gaacaaaaactcatctcagaag | Evan, 1994, Mol. Cell |
| | (SEQ ID NO: 15) | aggatctgaat | Biol. 5:3610 |
| | | (SEQ ID NO:16) | |
| Flag | DYKDDDDKG | gattacaaggacgacgatgac | Brizzard et al., 1994, |
| | (SEQ ID NO: 17) | aag | Biotechniques 16:730 |
| | | (SEQ ID NO: 18) | |
| His | HHHHHH | catcatcatcaccatcac | Synthetic |
| | (SEQ ID NO: 19) | (SEQ ID NO: 20) | |
| HA | YPYDVPDYA | tatccttatgatgttcctgattatg | Reverte et al., 2003, |
| | (SEQ ID NO: 21) | ca | Dev. Biol. 255:383 |
| | | (SEQ ID NO: 22) | |
| VSV-G | YTDIEMNRLGK | tatacagacatagagatgaacc | Kreis, 1986, EMBO J. |
| | (SEQ ID NO: 23) | gacttggaaag | 5:931 |
| | | (SEQ ID NO: 24) | |
| V5 | GKPIPNPLLGLDST | ggtaagcctatccctaaccctct | Southern et al., 1991, J. |
| | (SEQ ID NO: 25) | cctcggtctcgattctacg | Gen. Virol. 72:1551 |
| | | (SEQ ID NO: 26) | |
| HSV | QPELAPEDPED | cagcccgagctggcccccg | WO02/066675 |
| | (SEQ ID NO: 27) | aggaccccgaggac | |
| | | (SEQ ID NO: 28) | |

Detection of Tags

Tags that comprise an epitope for an antibody can be detected either in vivo or in vitro using anti-tag antibodies that are conjugated to a detectable marker. The detectable marker can be a naturally occurring or non-naturally occurring amino acid that bears, for example, radioisotopes (e.g., ¹²⁵I, ³⁵S), fluorescent or luminescent groups, biotin, haptens, antigens and enzymes. There are many commercially available Abs to tags, such as c-myc, HA, VSV-G, HSV, V5, His, and FLAG. In addition, antibodies to tags used in the invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology. 35: 1-21 (1990); and Kozbor et al., Immunology Today 4:72 (1983)). The anti-tag antibodies can then be detectably labeled through the use of radioisotopes, affinity labels (such as biotin, avidin, etc.), enzymatic labels (such as horseradish peroxidase, alkaline phosphatase, etc) using methods well known in the art, such as described in international application WO 00/70023 and (Harlour and Lane (1989) Antibodies, Cold Spring Harbor Laboratory, pp. 1-726), herein incorporated by reference.

Assays for detecting tags include, but are not limited to, Western Blot analysis, Immunohistochemistry, Elisa, FACS analysis, enzymatic assays, and autoradiography. Means for performing these assays are well known to those of skill in the art. For example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The tag can be further used to isolate the tag-immunoglobulin variable region polypeptide fusion protein away from other cellular material. For example, by immunoprecipitation, or by using anti-tag antibody affinity columns or anti-tag antibody conjugated beads. When a HIS tag is used, isolation can be performed using a metal-chelate column (See Hochuli in Genetic Engineering: Principles and Methods ed. J K Setlow, Plenum Press, NY, chp 18, pp 87-96). Means for performing these types of purification are well known in the art.

3. Immunoglobulin Variable Region Polypeptide Component

Herein, an immunoglobulin variable region includes i) an antibody heavy chain variable domain (V_{H}), or antigen binding fragment thereof, with or without constant region domains ii) an antibody light chain variable domain (V_{L}), or antigen binding fragment thereof, with or without constant region domains iii) a V_{H} or V_{L} domain polypeptide without constant region domains linked to another variable domain (a V_{H} or V_{L} domain polypeptide) that is with or without constant region domains, (e.g., V_{H}-V_{H}, V_{H}-V_{L}, or V_{L}-V_{L}), and iv) single-chain Fv antibodies (scFv), that is a V_{L} domain polypeptide without constant regions linked to another V_{H} domain polypeptide without constant regions (V_{H}-V_{L}), the variable domains together forming an antigen binding site. In one embodiment of option (i), (ii), or (iii), each variable domain forms and antigen binding site independently of any other variable domain. Option (i) or (ii) can be used to form a Fab fragment antibody or an Fv antibody. Thus, an immunoglobulin variable region polypeptide includes antibodies that may or may not contain constant region domains. In addition, immunoglobulin variable region polypeptides include antigen binding antibody fragments that can contain either all or just a portion of the corresponding heavy or light chain constant regions. In addition, an immunoglobulin variable region polypeptide include light chain, heavy chain, heavy and light chains (e.g., scFv), Fd (i.e., V_{H}-C_{H}1) or V_{L}-C_{L}. In addition, the term immunoglobulin variable region polypeptide can contain either all or just a portion of the corresponding heavy or light chain constant regions.

In a preferred embodiment the immunoglobulin variable region polypeptide is a single-domain antibody, or dAb. A single-domain antibody (dAb) refers to an immunoglobulin variable region polypeptide wherein antigen binding is effected by a single variable region domain. A single-domain antibody includes i) an antibody comprising heavy chain variable domain (V_{H}), or antigen binding fragment thereof, which forms an antigen binding site independently of any other variable domain, ii) an antibody comprising a light chain variable domain (V_{L}), or antigen binding fragment thereof, which forms an antigen binding site independently of any other variable domain, iii) an antibody comprising a V_{H} domain polypeptide linked to another V_{H} or a V_{L} domain polypeptide (e.g., V_{H}-V_{H} or V_{H}-V_{L}), wherein each V domain forms an antigen binding site independently of any other variable domain, and iv) an antibody comprising V_{L} domain polypeptide linked to another V_{L} domain polypeptide (V_{L}-V_{L}), wherein each V domain forms an antigen binding site independently of any other variable domain. As used herein, the V_{L} domain refers to both the kappa and lambda forms of the light chains.

To prepare nucleic acids encoding immunoglobulin variable region polypeptides, a source of genes encoding for antibodies is required. The genes can be obtained from natural sources (e.g., sources of rearranged or un-rearranged immunoglobulin genes) or synthetic sources. The source can be a heterogeneous population of antibody producing cells, for example B cells, preferably the source is rearranged B cells such as those found in the circulation or spleen of vertebrate. The source for genes encoding for the immunoglobulin region polypeptides can be biased, for example by obtaining B cells from vertebrates in any one of various stages of age, health and immune response (e.g., from an animal that has been preimmunized by a defined antigen). Nucleic acids coding for immunoglobulin variable region polypeptides can also be derived from other cells producing IgA, IgD, IgE, or IgM.

Methods for preparing fragments of genomic DNA where immunoglobulin variable regions can be cloned as a diverse population are well known in the art. See for example Hermann et al., Methods in Enzymology, 152: 180-183, (1987); Frischauf, Methods in Enzymology, 152:183-190 (1987); Frischauf, Methods in Enzymology, 152:190-199 (1987); and DiLella et al., Methods in Enzymology, 152: 199-212 (1987), the teachings of which are herein incorporated by reference. Briefly, rearranged immunoglobulin genes can be cloned from genomic DNA or mRNA. For the latter, mRNA is extracted from the cells and the cDNA is prepared using reverse transcriptase and poly dT oligonucleotide primers. Primers for cloning sequences encoding antibodies are discussed by Larrick, et al., Bio/Technology 7:934 (1989), and Danielsson & Borrebaceick, in Antibody Engineering: a practical guide (Freeman, N.Y., 1992), pg 89 and Huse, id. at chapter 5.

Diversity of the immunoglobulin variable region polypeptides can arise from obtaining antibody-encoding sequences from a natural source, such as a non-clonal population of immunized or non-immunized B cells. Alternatively, or additionally, diversity can be introduced by artificial mutagenesis, see section C of this application entitled “Mutageneisis using polymerase chain reaction (PCR)”.

According to the invention, the residues which are varied to obtain diversity are a subset of those that form the binding site for the target ligand. Different (including overlapping) subsets of residues in the target ligand binding site can be diversified at different stages during selection, if desired. The diversification of chosen positions is achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK, which encodes all amino acids as well as the TAG stop codon. The NNK codon is preferably used in order to introduce the required diversity. Other codons that achieve the same ends are also of use, including the NNN codon, which leads to the production of the additional stop codons TGA and TAA. Means for generating antibody libraries and diversity using NNK and NNN codons are described in International patent application WO 99/20749, herein fully incorporated by reference.

4. Bacteriophage Coat Protein Component

In the present invention, a variety of bacteriophage systems and bacteriophage coat proteins can be used. Examples of suitable bacteriophage coat proteins include, without limitation, M13 gene III, gene VIII; rd minor coat protein pIII (Saggio et al, Gene 152:35, 1995); lambda D protein (Sternberg & Hoess, Proc. Natl. Acad. Sci. USA 92:1609, 1995; Mikawa et al., J. Mol. Biol. 262:21, 1996); lambda phage tail protein pV (Maruyama et al., Proc. Natl. Acad. Sci. USA 91:8273, 1994; U.S. Pat. No. 5,627,024); fr coat protein (WO96/11947; DD 292928; DD 286817; DD 300652); Φ29 tail protein gp9 (Lee, Virol. 69:5018, 1995); MS2 coat protein; T4 small outer capsid protein (Ren et al., Protein Sci. 5:1833, 1996), T4 nonessential capsid scaffold protein IPIII (Hong and Black, Virology 194:481, 1993), or T4 lengthened fibritin protein gene (Efimov, Virus Genes 10:173, 1995); PRD-1 gene III; Qβ3 capsid protein (as long as dimerization is not interfered with); and P22 tailspike protein (Carbonell and Villayerde, Gene 176:225, 1996). Techniques for inserting foreign coding sequence into a phage gene are well known (see e.g., Sambrook et al., Molecular Cloning: A Laboratory Approach, Cold Spring Hargor Press, NY, 1989; Ausubel et al., Current Protocols in Molecular Biology, Greene Publishing Co., NY, 1995).

In a preferred aspect of the invention a filamentous bacteriophage coat protein is used. Many filamentous bacteriophage vectors are commercially available that can allow for the in-frame ligation of the signal peptide-tag-immunoglobulin variable region polypeptide fusion protein to a bacteriophage coat protein. The most common vectors accept DNA inserts for in frame fusions with gene III or gene VIII. Non-limiting examples of suitable vectors include, M13 mp vectors (Pharmacia Biotech), pCANTAB 5e (Pharmacia Biotech), pCOMB3 and M13KE (New England Biolabs), pBluescript series (Stratagene Cloning Systems, La Jolla, Calif.). It should be understood that these vectors already contain bacteriophage signal peptide sequences and that each vector can be modified to contain the bacteriophage signal peptide sequence of interest by methods well known in the art (Sambrook et al., Molecular Biology: A laboratory Approach, Cold Spring Harbor, N.Y. 1989; Ausubel, et al., Current protocols in Molecular Biology, Greene Publishing, Y, 1995

5. Construction of Vectors

Herein, a tag sequence is fused in frame in between a bacteriophage signal sequence and an immunoglobulin variable region polypeptide, which can be fused in frame to a bacteriophage protein. The vectors can be constructed using standard methods (Sambrook et al., Molecular Biology: A laboratory Approach, Cold Spring Harbor, N.Y. 1989; Ausubel, et al., Current protocols in Molecular Biology, Greene Publishing, Y, 1995), guided by the principles discussed below. In brief, conventional ligation techniques are used to insert DNA sequences encoding the signal peptide, tag, and immunoglobulin variable region polypeptide into an expression vector, in the following order: signal peptide-tag-immunoglobulin variable region. The sequences are ligated such that the components are expressed as an in-frame fusion. In one embodiment, the DNA encoding the signal peptide/tag/immunoglobulin variable region polypeptide is further ligated in frame to a bacteriophage coat protein in order that the immunoglobulin variable region polypeptide fusion protein can be displayed on the surface of a bacteriophage particle.

In another aspect of the invention, a DNA sequence encoding a single-domain antibody is linked at its 5′ end to a DNA sequence encoding a signal peptide, in the absence of a tag using standard molecular biology techniques referenced above. In one embodiment the DNA encoding the signal peptide/single/domain antibody fusion protein is further ligated in frame to a bacteriophage coat protein in order that the single-domain antibody can be displayed on the surface of a bacteriophage particle.

Vectors and Host cells

The manipulation of nucleic acids in the present invention is typically carried out in recombinant vectors. Herein, both phagemid and non-phagemid vectors can be used. As used herein, vector refers to a discrete element that is used to introduce heterologous DNA into cells for the expression and/or replication thereof. Methods by which to select or construct and, subsequently, use such vectors are well known to one of skill in the art. Numerous vectors are publicly available, including bacterial plasmids, bacteriophage, artificial chromosomes, episomal vectors and gene expression vectors can be employed. A vector of use according to the invention may be selected to accommodate a polypeptide coding sequence of a desired size. A suitable host cell is transformed with the vector after in vitro cloning manipulations. Host cells may be prokaryotic, such as any of a number of bacterial strains, or may be eukaryotic, such as yeast or other fungal cells, insect or amphibian cells, or mammalian cells including, for example, rodent, simian or human cells. Each vector contains various functional components, which generally include a cloning (or “polylinker”) site, an origin of replication and at least one selectable marker gene. If given vector is an expression vector, it additionally possesses one or more of the following: enhancer element, promoter, transcription termination and signal sequences, each positioned in the vicinity of the cloning site, such that they are operatively linked to the gene encoding a polypeptide repertoire member according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. For example, the origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as a selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Since the replication of vectors according to the present invention is most conveniently performed in E. coli (e.g., strain TB1 or TG1), an E. coli-selectable marker, for example, the β-lactamase gene that confers resistance to the antibiotic ampicillin, is of use. These can be obtained from E. Coli plasmids, such as pBR322 or a pUC plasmid such as pUC18 or pUC19, or pUC119.

Expression vectors usually contain a promoter that is recognized by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the α-lactamase and lactose promoter systems, alkaline phosphatase, the tryptophan (trp) promoter system and hybrid promoters such as the tac promoter. Promoters for use in bacterial systems will also generally contain a Shine-Delgamo sequence operably linked to the coding sequence. Preferred promoters for use in the present invention are the isopropylthiogalactoside (IPTG)-regulatable promoters.

In a preferred aspect of the invention a filamentous bacteriophage vector system is used for expression of the signal peptide/tag/immunoglobulin variable region polypeptide fusion protein, or the signal-peptide/single-domain antibody fusion protein in order that the fusion proteins can be incorporated into bacteriophage for display on the outer surface of the bacteriophage particle. Many filamentous bacteriophage vectors (phage vectors) are commercially available for use that allow for the in-frame ligation of the DNA encoding the immunoglobulin variable region polypeptide fusion protein to a bacteriophage coat protein. The most common vectors accept DNA inserts for in frame fusions with gene III or gene VIII. Non-limiting examples of suitable vectors include, M13 mp vectors (Pharmacia Biotech), pCANTAB 5e (Pharmacia Biotech), pCOMB3 and M13KE (New England Biolabs), and others as described in WO 00/29555, herein incorporated by reference. It should be understood that these vectors already contain bacteriophage signal peptide sequences and that each vector can be modified to contain the bacteriophage signal peptide sequence of interest by methods well known in the art (Sambrook et al., Molecular Biology: A laboratory Approach, Cold Spring Harbor, N.Y. 1989; Ausubel, et al., Current protocols in Molecular Biology, Greene Publishing, Y, 1995).

Introduction of Vectors to Host Cells.

Vectors useful in the present invention may be introduced to selected host cells by any of a number of suitable methods known to those skilled in the art. For example, vector constructs may be introduced to appropriate bacterial cells by infection, in the case of E. coli bacteriophage vector particles such as lambda or M13, or by any of a number of transformation methods for plasmid vectors or for bacteriophage DNA. For example, standard calcium-chloride-mediated bacterial transformation is still commonly used to introduce naked DNA to bacteria (Sambrook et al., 1989, Molecular Cloning, A Laboratory Manual, Cold Spring Harbor Laboratory Press, Cold Spring Harbor, N.Y.), but electroporation may also be used (Ausubel et al., 1988, Current Protocols in Molecular Biology, (John Wiley & Sons, Inc., NY, N.Y.)).

For the introduction of vector constructs to yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisiae, for example, the cells are treated with lithium acetate to achieve transformation efficiencies of approximately 104 colony-forming units (transformed cells)/μg of DNA. Transformed cells are then isolated on selective media appropriate to the selectable marker used. Alternatively, or in addition, plates or filters lifted from plates may be scanned for GFP fluorescence to identify transformed clones.

For the introduction of vectors comprising differentially expressed sequences to mammalian cells, the method used will depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation. These methods are detailed, for example, in Current Protocols in Molecular Biology (Ausubel et al., 1988, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

B. Construction of Libraries

In one aspect of the invention is the generation of nucleic acid and polypeptide libraries using the immunoglobulin variable region polypeptide fusion proteins described herein. As used herein, the term “library” refers to a mixture of heterogeneous polypeptides or nucleic acids. The library is composed of members, a plurality of which has a unique polypeptide or nucleic acid sequence. To this extent, library is synonymous with repertoire. Sequence differences between library members are responsible for the diversity present in the library. The library can take the form of a simple mixture of polypeptides or nucleic acids, or can be in the form organisms or cells, for example bacteria, viruses, animal or plant cells and the like, transformed with a library of nucleic acids. Typically, each individual organism or cell contains only one member of the library. In certain applications, each individual organism or cell can contain two or more members of the library. Advantageously, the nucleic acids are incorporated into expression vectors, in order to allow expression of the polypeptides encoded by the nucleic acids. In a preferred aspect, therefore, a library can take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

A number of vector systems useful for library production and selection are known in the art. For example, bacteriophage lambda expression systems can be screened directly as bacteriophage plaques or as colonies of lysogens, both as previously described (Huse et al., Science, 246:1275-1281, 1989; Caton & Koprowski, Proc. Natl. Acad. Sci. USA, 87:6450-6454, 1990; Mullinax et al., Proc. Natl. Acad. Sci. USA, 87:8095-8099, 1990; Persson et al., Proc. Natl. Acad. Sci. USA, 88:2432-2436, 1991) and are of use in the invention. While such expression systems can be used for screening up to 10⁶ different members of a library, they are not really suited to screening of larger numbers (greater than 10⁶ members). Other screening systems rely, for example, on direct chemical synthesis of library members. One early method involves the synthesis of peptides on a set of pins or rods, such as described in WO84/03564. A similar method involving peptide synthesis on beads, which forms a peptide library in which each bead is an individual library member, is described in U.S. Pat. No. 4,631,211 and a related method is described in WO92/00091. A significant improvement of the bead-based methods involves tagging each bead with a unique identifier tag, such as an oligonucleotide, so as to facilitate identification of the amino acid sequence of each library member. These improved bead-based methods are described in WO93/06121.

Another chemical synthesis method involves the synthesis of arrays of peptides (or peptidomimetics) on a surface in a manner that places each distinct library member (e.g., unique peptide sequence) at a discrete, predefined location in the array, or the spotting of pre-formed polypeptides on such an array. The identity of each library member is determined by its spatial location in the array. The locations in the array where binding interactions between a predetermined molecule (e.g., a receptor) and reactive library members occur is determined, thereby identifying the sequences of the reactive library members on the basis of spatial location. These methods are described in U.S. Pat. No. 5,143,854; WO 90/15070 and WO 92/10092; Fodor et al., Science, 251:767-773, 1991; and Dower & Fodor, Ann. Rep. Med. Chem., 26:271-280, 1991).

Of particular use in the construction of libraries of the invention are selection display systems, which enable a nucleic acid to be linked to the polypeptide it expresses. As used herein, a selection display system is a system that permits the selection, by suitable display means, of the individual members of the library.

Any selection display system can be used in conjunction with a library according to the invention. For example, immunoglobulin variable region polypeptide fusion proteins of the present invention may be displayed on lambda phage capsids (phage bodies). Preferred selection systems of the invention are the filamentous bacteriophage systems. Selection protocols for isolating desired members of large libraries are known in the art, as typified by phage display techniques. An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phage vector, sequencing, expression and subsequent genetic manipulation is relatively straightforward.

Methods for the construction of bacteriophage antibody display libraries and lambda phage expression libraries are well known in the art (McCafferty et al., Nature, 348:552-554, 1990; Kang et al., Proc. Natl. Acad. Sci. USA, 88:11120-11123, 1991; Clackson et al., Nature, 352:624-628, 1991; Lowman et al., Biochemistry, 30:10832-10838, 1991; Burton et al., Proc. Natl. Acad. Sci. USA, 88:10134-10137, 1991; Hoogenboom et al., Nucleic Acid Res., 19:4133-4137, 1991; Chang et al., J. Immunol., 147:3610-3614, 1991; Breitling et al., Gene, 104:147-153, 1991; Marks et al., J. Biol. Chem., 267:16007-16010, 1991; Barbas et al., Proc. Natl. Acad. Sci. USA, 89:10164-10168, 1992; Hawkins & Winter, Eur. J. Immunol., 22:867-870, 1992; Marks et al., J. Biol. Chem., 267:16007-16010, 1992; Lemer et al., Science, 258:1313-1314, 1992, incorporated herein by reference). In brief, the nucleic acids encoding the immunoglobulin variable region polypeptide fusion proteins are cloned into a phage vector that comprises a bacteriophage packaging signal and a gene encoding at least one bacteriophage coat protein which allows for the incorporation of the nucleic acid into a phage particle.

Other systems for generating libraries of polypeptides or polynucleotides involve the use of cell-free enzymatic machinery for the in vitro synthesis of the library members. For example, in vitro translation can be used to synthesize polypeptides as a method for generating large libraries. These methods are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax), use the polysomes to display polypeptides for selection. These and all the foregoing documents are incorporated herein by reference.

Immunoglobulin variable region antibody libraries according to the present invention may advantageously be designed to be based on a predetermined main chain conformation. Such libraries may be constructed as described in International Patent Application WO 99/20749, the contents of which are incorporated herein by reference. Thus, in one embodiment of the invention, the immunoglobulin variable region polypeptide or single-domain antibody comprises an antibody heavy chain variable region polypeptide or single-domain antibody comprising an antibody heavy chain variable domain (V_{H}), or antigen binding fragment thereof, which comprises the amino acid sequence of germline V_{H} segment DP-47. In another embodiment of the invention, the immunoglobulin variable region polypeptide or single-domain antibody comprises an antibody light chain variable domain (V_{L}), or antigen binding fragment thereof, which comprises the amino acid sequence of germline V_{κ} segment DPK9. Such variable region polypeptides can be used for the production of scFvs or Fabs, e.g., an scFv or Fab comprising (i) an antibody heavy chain variable domain (V_{H}), or antigen binding fragment thereof, which comprises the amino acid sequence of germline V_{H} segment DP-47 and (ii) an antibody light chain variable domain (V_{L}), or antigen binding fragment thereof, which comprises the amino acid sequence of germline V_{κ} segment DPK9.

C. Library Diversity

Mutagenesis Using the Polymerase Chain Reaction (PCR)

Once nucleic acid sequences encoding members of the polypeptide repertoire are cloned into the vector, one may generate diversity within the cloned molecules by undertaking mutagenesis prior to expression. Mutagenesis of nucleic acid sequences encoding polypeptide repertoires is carried out by standard molecular methods. Of particular use is the polymerase chain reaction, or PCR, (Mullis and Faloona (1987) Methods Enzymol., 155: 335, herein incorporated by reference). PCR, which uses multiple cycles of DNA replication catalysed by a thermostable, DNA-dependent DNA polymerase to amplify the target sequence of interest, is well known in the art.

Oligonucleotide primers useful according to the invention are single-stranded DNA or RNA molecules that hybridize selectively to a nucleic acid template to prime enzymatic synthesis of a second nucleic acid strand. The primer is complementary to a portion of a target molecule present in a pool of nucleic acid molecules used in the preparation of sets of arrays of the invention. It is contemplated that such a molecule is prepared by synthetic methods, either chemical or enzymatic. Alternatively, such a molecule or a fragment thereof is naturally occurring, and is isolated from its natural source or purchased from a commercial supplier. Mutagenic oligonucleotide primers are 15 to 100 nucleotides in length, ideally from to 40 nucleotides, although oligonucleotides of different length are of use.

Typically, selective hybridization occurs when two nucleic acid sequences are substantially complementary (at least about 65% complementary over a stretch of at least 14 to 25 nucleotides, preferably at least about 75%, more preferably at least about 90% complementary). See Kanehisa (1984) Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, it may comprise nucleotide loops, which we define as regions in which mismatch encompasses an uninterrupted series of four or more nucleotides.

Overall, five factors influence the efficiency and selectivity of hybridization of the primer to a second nucleic acid molecule. These factors, which are (i) primer length, (ii) the nucleotide sequence and/or composition, (iii) hybridization temperature, (iv) buffer chemistry and (v) the potential for steric hindrance in the region to which the primer is required to hybridise, are important considerations when non-random priming sequences are designed.

There is a positive correlation between primer length and both the efficiency and accuracy with which a primer will anneal to a target sequence: longer sequences have a higher melting temperature (TM) than do shorter ones, and are less likely to be repeated within a given target sequence, thereby minimizing promiscuous hybridization. Primer sequences with a high G-C content or that comprise palindromic sequences tend to self-hybridise, as do their intended target sites, since unimolecular, rather than bimolecular, hybridization kinetics are generally favored in solution: at the same time, it is important to design a primer containing sufficient numbers of G-C nucleotide pairings to bind the target sequence tightly, since each such pair is bound by three hydrogen bonds, rather than the two that are found when A and T bases pair. Hybridization temperature varies inversely with primer annealing efficiency, as does the concentration of organic solvents, e.g. formamide, that might be included in a hybridization mixture, while increases in salt concentration facilitate binding. Under stringent hybridization conditions, longer probes hybridize more efficiently than do shorter ones, which are sufficient under more permissive conditions. Stringent hybridization conditions typically include salt concentrations of less than about 1M, more usually less than about 500 mM and preferably less than about 200 mM. Hybridization temperatures range from as low as 0° C. to greater than 22° C., greater than about 30° C., and (most often) in excess of about 37° C. Longer fragments may require higher hybridization temperatures for specific hybridization. As several factors affect the stringency of hybridization, the combination of parameters is more important than the absolute measure of any one alone.

Primers preferably are designed using computer programs that assist in the generation and optimization of primer sequences. Examples of such programs are “PrimerSelect” of the DNAStar™ software package (DNAStar. Inc.; Madison, Wis.) and OLIGO 4.0 (National Biosciences. Inc.). Once designed, suitable oligonucleotides are prepared by a suitable method, e.g. the phosphoramidite method described by Beaucage and Carruthers (1981) Tetrahedron Lett., 22: 1859) or the triester method according to Matteucci and Caruthers (1981) J. Am. Chem. Soc., 103: 3185, both incorporated herein by reference, or by other chemical methods using either a commercial automated oligonucleotide synthesiser or VLSIPS™ technology.

PCR is performed using template DNA (at least lfg: more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 pmol of oligonucleotide primer, 2.5 μl of 10× PCR buffer 1 (Perkin-Elmer, Foster City, Calif.), 0.4μ of 1.25 mM dNTP, 0.15 μl (or 2.5 units) of Taq DNA polymerase (Perkin Elmer, Foster City, Calif.) and deionised water to a total volume of 25 μl. Mineral oil is overlaid and the PCR is performed using a programmable thermal cycler.

The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenised, mismatch is required, at least in the first round of synthesis. In attempting to amplify a population of molecules using a mixed pool of mutagenic primers, the loss, under stringent (high-temperature) annealing conditions, of potential mutant products that would only result from low melting temperatures is weighed against the promiscuous annealing of primers to sequences other than the target site. The ability to optimise the stringency of primer annealing conditions is well within the knowledge of one of skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above: 1-2 minutes), and extension (72° C. for 1-5 minutes, depending on the length of the amplified product). Final extension is generally for 4 minutes at 72° C., and may be followed by an indefinite (0-24 hour) step at 4° C.

One PCR based method to generate diversity involves the use of random degenerative oligonucleotides. In a preferred embodiment, residues which are varied by PCR to obtain diversity are a subset of those that form the binding site for the target ligand. Different (including overlapping) subsets of residues in the target ligand binding site can be diversified at different stages during selection, if desired. The diversification of chosen positions is achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK (N is any nucleotide A, C, G, or T and K is T or G), which encodes all amino acids as well as the TAG stop codon. The NNK codon is preferably used in order to introduce the required diversity. Other codons that achieve the same ends are also of use, including the NNN codon, which leads to the production of the additional stop codons TGA and TAA. Means for generating antibody libraries with diversity using NNK and NNN codons are described in International patent application WO 99/20749, herein fully incorporated by reference.

After generation of diversity through PCR, the double stranded PCR fragments are cloned into appropriate vectors for generation of the libraries described under the heading library construction. In a preferred embodiment the PCR fragments are cloned into a phage vector for generation of a bacteriophage library that encodes a repertoire of immunoglobulin variable region polypeptide fusion proteins

D. Selection/Screening Systems According to the Invention

The invention provides a method for selecting from a repertoire of polypeptides, a population of immunoglobulin variable region polypeptides that bind a target ligand comprising contacting the polypeptide library with a target ligand and selecting a population of polypeptides which bind to the target ligand. The peptide libraries according to the invention can be screened in a selection protocol that involves a genetic display package, e.g. phage display. Alternatively the peptide library can be screened in the absence of a genetic display package, for example, using peptides displayed on an array or beads.

Phage Display

In a preferred embodiment the bacteriophage signal sequence/tag/immunoglobulin variable region polypeptide fusion protein, or the signal peptide/immunoglobulin variable region polypeptide fusion protein is additionally fused to a bacteriophage coat protein for phage display. A sequence encoding the immunoglobulin variable region polypeptide fusion proteins of the invention are engineered adjacent to a gene encoding a bacteriophage coat protein as to display the ligand on the outer surface of the bacteriophage particle. In general, E. Coli. is transformed with a library of phage vectors that encode the repertoire of immunoglobulin variable region polypeptide fusion proteins. In one aspect of the present invention, a library of nucleotide sequences encoding immunoglobulin variable region polypeptides is fused in frame to, a bacteriophage signal peptide coding sequence, a tag coding sequence, and a bacteriophage coat protein coding sequence in the following order (5′ to 3′): bacteriophage signal peptide-tag-immunoglobulin variable region-bacteriophage coat protein. The bacteriophage signal sequence directs the expressed immunoglobulin variable region polypeptide fusion protein to the periplasmic space which allows for incorporation of the immunoglobulin variable region polypeptide fusion protein into bacteriophage particles and its display on the bacteriophage particle surface. The bacteriophage library is then screened for specific binding to target ligand by methods well known in the art, (Abelson, J. and Simon, M. (eds), Methods in Enzymology, combinatorial chemistry, Vol. 267, San Diego: Academic Press (1996), Kay, B. K., Winter, J., McCafferty, J. (eds), Phage Display of peptides and Proteins, A laboratory Manual, San Diego: Academic Press (1996)), each incorporated herein by reference.

The strategy is to enrich for library members that bind to target ligand by performing successive rounds of affinity selection and amplification of bound bacteriophage particles, a process known a panning (Parmely & Smith, Gene 73: 305-318 (1988)). Briefly, the library of phage particles is incubated with target ligand. The target ligand can be immobilized on a surface or particle, optionally anchored by a tether (3 to 12 carbons, for example) to hold the target far enough away from the surface to permit free interaction of the target with the immunoglobulin variable region polypeptide fusion protein. An example of how the target ligand can be immobilized is through a streptavidin-biotin interaction. The target should lack specific binding affinity for the tag, because it is the bacteriophage displayed immunoglobulin-variable region polypeptide that is being screened for binding to target and not the tag. The conditions for incubation of the phage library with the target ligand can vary, however in all cases the binding reaction is allowed to reach equilibrium.

The unbound library members are then washed away from the immobilized target. The degree and stringency of washing can be varied. For example, the temperature, pH, ionic strength, divalent cation concentration, volume and duration of the washing can be varied to select for immunglobulin variable region polypeptides that have different affinities for the target ligand. A selection based on a high affinity interaction is preferred. High affinity interactions are obtained by adding a saturating amount of free target ligand, or by increasing the volume, number and length of washes. This prevents the re-binding of disassociated phage particles, and immunoglobulin variable region polypeptides of higher and higher affinity are recovered.

After washing at the appropriate stringency, the bound bacteriophage particles are eluted from the immobilized target by exposing them to a pH shift. For example, pH 2 or pH 11 can be used. The pH is then neutralized and the eluted phage are amplified by infecting or transforming host cells, for example E. coli, using an appropriate selection marker, e.g., antibiotic. The bacteriophage produced by the host cells are used in another round of affinity selection (panning), and the cycle is repeated until the desired level enrichment is achieved.

To isolate the individual clones, bacteriophage particles from the final round of panning are infected into host cells, alternatively their DNA is transformed into host cells and the cells are grown on LB-agar plates in the presence of an appropriate selection marker, e.g., antibiotic. Each colony represents an individual DNA sequence encoding an immunoglobulin variable region polypeptide.

The replicative form of the bacteriophage DNA in each colony can be isolated by standard means. The immunoglobulin variable region polypeptide fusion protein can then be cloned into an eukaryotic or prokaryotic expression vector for the expression of soluble polypeptide without the bacteriophage coat protein. In one embodiment, the original phage vector contains an Amber stop codon upstream of the sequence encoding bacteriophage coat protein. Thus, in this embodiment the immunoglobulin variable region polypeptide fusion protein does not need to be cloned into an expression vector for the expression of soluble peptide. The original phage vector need only be transformed into an appropriate host cell that does not contain an Amber suppressor, for example, E. coli strain TB1. Expression of the immunoglobulin variable region polypeptide in such cells allow for the expression of the signal peptide/tag/immunoglobulin variable region polypeptide, or signal peptide/immunoglobulin variable region polypeptide fusion proteins without the bacteriophage coat protein.

It is preferred that the immunoglobulin variable region polypeptide is expressed in bacteria wherein the polypeptide can be isolated from the bacterial periplasm by methods well known in the art. Preferred bacteria of use are E. coli strains TG1 and TB1, most preferably, TB1 Briefly, the immunoglobulin variable region polypeptide fusion proteins of the present invention are allowed to accumulate in the periplasmic space of bacteria by growing a transformed bacterial culture at 37° C. for approximately 3-5 hours. The bacterial cell wall is lysed by cold osmotic shock (Tris-EDTA) and then rapidly diluted in a chilled solution of low osmotic strength (Tris-EDTA/H₂O). The EDTA makes the outer membrane more permeable, and the cold inhibits protease activity. The lysed bacterial cells are then centrifuged and the supernatant contains the periplasmic proteins.

The periplasmic proteins can then be directly tested by ELISA to detect and quantitate the presence of the immunoglobulin variable region polypeptide. The target molecule used in the ELISA can be either an anti-tag antibody, or another known ligand of the tag. Alternatively, the target ligand used in the ELISA can be the same target ligand used in the screening assay.

The immunoglobulin variable region polypeptide fusion proteins can further be isolated from the other periplasmic proteins by a variety of purification methods such as, immunoprecipitation, affinity chromatography, and the like.

Other Screening Methods

Herein, screening methods and systems other than phage display can be used. These systems comprise the display of the immunoglobulin variable region fusion proteins on a solid support, for example display on a bead or on an array. For example, each distinct library member (e.g., unique peptide sequence) may be placed at a discrete, predefined location in the array and the identity of each library member is determined by its spatial location in the array. Methods for screening using such an array are described in U.S. Pat. No. 5,143,854; WO90/15070 and WO92/10092; Fodor et al., Science, 251:767-773, 1991; and Dower & Fodor, Ann. Rep. Med. Chem., 26:271-280, 1991), herein incorporated by reference. Briefly, to screen the immunoglobulin variable region polypeptide fusion proteins described herein for the ability to bind target ligand, the arrayed proteins are contacted with a ligand that comprises a detectable marker, (e.g., a fluorescent or radioactive label), or with a ligand that can be detected by a labeled antibody. The location of the marker on the substrate array is detected with, for example photon detection or autoradiographic techniques. The DNA sequence of the immunoglobulin variable region polypeptide fusion protein that binds target ligand can then be easily detected using the predetermined knowledge of the DNA sequence of the material at the location where binding is detected.

Methods for screening using polysomal display are well known in the art and are described in WO95/22625 and WO95/11922 (Affymax), which are herein incorporated by reference in their entirety. Briefly, the displayed variable domain region polypeptides are selected from the library by an affinity enrichment technique similar to phage display described above. Target ligand is immobilized on a solid support and a repeat affinity selection procedure is performed. The target ligand is contacted with library members and the members that do not bind target ligand are removed by washing. The stringency of the wash can be adjusted to provide a certain degree of control over the binding characteristics. The polysome is a peptide/polynucleotide complex, to obtain the nucleotide sequence of the binder, a high stringency wash is performed and the nucleic acid is amplified by PCR.

D. Use of Polypeptides Selected According to the Invention

Polypeptides selected according to the method of the present invention may be employed in substantially any process which involves ligand-polypeptide binding, including in vivo therapeutic and prophylactic applications, in vitro and in vivo diagnostic applications, in vitro assay and reagent applications, and the like. For example, the immunoglobulin variable region polypeptide molecules may be used in antibody based assay techniques, such as ELISA techniques, according to methods known to those skilled in the art.

The molecules selected according to the invention are of further use in diagnostic, prophylactic and therapeutic procedures. For example, immunoglobulin variable region polypeptides selected according to the invention are of use diagnostically in Western analysis and in situ protein detection by standard immunohistochemical procedures. In addition, such immunoglobulin variable region polypeptides may be used preparatively in affinity chromatography procedures, when complexed to a chromatographic support, such as a resin. All such techniques are well known to one of skill in the art.

Therapeutic and prophylactic uses of proteins prepared according to the invention involve the administration of the immunoglobulin variable region polypeptides selected according to the invention to a recipient mammal, such as a human, and subsequent interaction of immunoglobulin variable region polypeptide with target ligand.

Substantially pure antibodies of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981). Immunological Methods, Volumes I and II, Academic Press, NY).

The selected antibodies of the present invention will typically find use in preventing, suppressing or treating inflammatory states, allergic hypersensitivity, asthma, cancer, bacterial or viral infection, and autoimmune disorders (which include, but are not limited to, Type I diabetes, multiple sclerosis, rheumatoid arthritis, systemic lupus erythematosus, Crohn's disease and myasthenia gravis).

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest to eradicate or reduce the disease symptoms.

Animal model systems which can be used to screen the effectiveness of the immunoglobulin variable region polypeptide in protecting against or treating the disease are available. Methods for the testing of systemic lupus erythematosus (SLE) in susceptible mice are known in the art (Knight et al. (1978) J. Rip. Med., 147: 1653; Reinersten et al. (1978) New Eng. J. Med., 299: 515). Myasthenia Gravis (MG) is tested in SJL/J female mice by inducing the disease with soluble AchR protein from another species (Lindstrom et al. (1988) Adv. Immunol., 42: 233). Arthritis is induced in a susceptible strain of mice by injection of Type II collagen (Stuart et al. (1984) Ann. Rev. Immunol., 42: 233). A model by which adjuvant arthritis is induced in susceptible rats by injection of mycobacterial heat shock protein has been described (Van Eden et al. (1988) Nature, 331: 171). Thyroiditis is induced in mice by administration of thyroglobulin as described (Maron et al. (1980) J. Exp. Med., 152: 1115). Insulin dependent diabetes mellirus (IDDM) occurs naturally or can be induced in certain strains of mice such as those described by Kanasawa et al. (1984) Diabetologia, 27: 113. EAE in mouse and rat serves as a model for MS in human. In this model, the demyelinating disease is induced by administration of myelin basic protein (see Paterson (1986). Textbook of Immunopathology, Mischer et al., eds., Grune and Stratton, N.Y., pp. 179-213; McFarlin et al. (1973) Science, 179: 478; and Satoh et al. (1987) J. Immunol., 138: 179). In addition, numerous animal models useful for the study of asthma are known in the art and which may be useful according to the present invention (See, e.g., U.S. Pat. Nos. 6,284,800; 5,730,983; Isenberg-Feig et al., 2003 “Animal Models of Allergic Asthma”, Curr. Allergy Asthma Rep. 3:70).

The selected immunoglobulin variable region polypeptide of the present invention may also be used in combination with other antibodies, particularly monoclonal antibodies (MAbs) reactive with other markers on human cells responsible for the diseases. For example, suitable T-cell markers can include those grouped into the so-called “Clusters of Differentiation,” as named by the First International Leukocyte Differentiation Workshop (Bernhard et al. (1984). Leukocyte Typing, Springer Verlag, N.Y.).

Generally, the present selected single domain antibodies will be utilized in purified form together with pharmacologically appropriate carriers. Typically, these carriers include aqueous or alcoholic/aqueous solutions, emulsions or suspensions, any including saline and/or buffered media, Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982). Remington's Pharmaceutical Sciences, 16th Edition).

The selected immunoglobulin variable region polypeptide or single-domain antibodies of the present invention may be used as separately administered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with the selected antibodies, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, including without limitation immunotherapy, the selected immunoglobulin variable domain polypeptides or single-domain antibodies of the invention can be administered to any patient in accordance with standard techniques. The administration can be by any appropriate mode, including parenterally, intravenously, intramuscularly, intraperitoneally, transdermally, via the pulmonary route, or by direct infusion with a catheter. The dosage and frequency of administration will depend on the age, sex and condition of the patient, concurrent administration of other drugs, contra-indications and other parameters to be taken into account by the clinician.

The selected immunoglobulin variable region polypeptide or single-domain antibodies of this invention can be lyophilized for storage and reconstituted in a suitable carrier prior to use. This technique has been shown to be effective with conventional immunoglobulins and art-known lyophilization and reconstitution techniques can be employed. It will be appreciated by those skilled in the art that lyophilization and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the present immunoglobulin variable region polypeptide or single-domain antibodies or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. A “therapeutically effective dose” also refers to an amount effective to reduce one or more symptoms of a disease. Amounts needed to achieve this dosage will depend upon the severity of the disease and the general state of the patient's own immune system, but generally range from 0.005 to 5.0 mg of selected antibody, receptor (e.g. a T-cell receptor) or binding protein thereofper kilogram of body weight, with doses of 0.05 to 2.0 mg/kg/dose being more commonly used. For prophylactic applications, compositions containing the present selected polypeptides or cocktails thereof may also be administered in similar or slightly lower dosages.

A composition containing a selected immunoglobulin variable region polypeptide or single-domain antibody according to the present invention may be utilized in prophylactic and therapeutic settings to aid in the alteration, inactivation, killing or removal of a select target cell population in a mammal. In addition, the selected repertoires of single-domain antibodies described herein may be used extracorporeally or in vitro selectively to kill, deplete or otherwise effectively remove a target cell population from a heterogeneous collection of cells. Blood from a mammal may be combined extracorporeally with the selected single-domain antibodies, whereby the undesired cells are killed or otherwise removed from the blood for return to the mammal in accordance with standard techniques.

The invention is further described, for the purposes of illustration only, in the following examples.

### EXAMPLES

To test the feasibility of using the N-terminal FLAG tag and new multiple cloning site in a phage vector for cloning dAb libraries, V_{H} and V_{κ} dAbs were cloned into this newly created vector. Phage particles of two V_{H} dAbs (V_{H} dummy and HEL4) and two V_{K} dAbs (V_{K} dummy and BSA28) were produced and tested for their ability to bind protein A or L or antigen in ELISA.

Subsequently a phage library was constructed and selections were performed against 3 antigens: Antigen #1 (hen egg lysosyme) and human and mouse versions of another antigen (Antigens #2 and #3 respectively). Selections against antigens were successful.

Methods

Construction of Phage Vector:

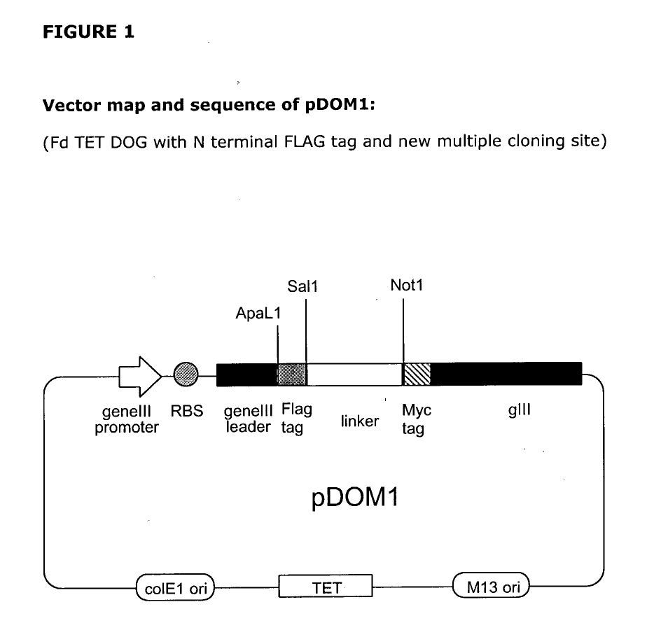

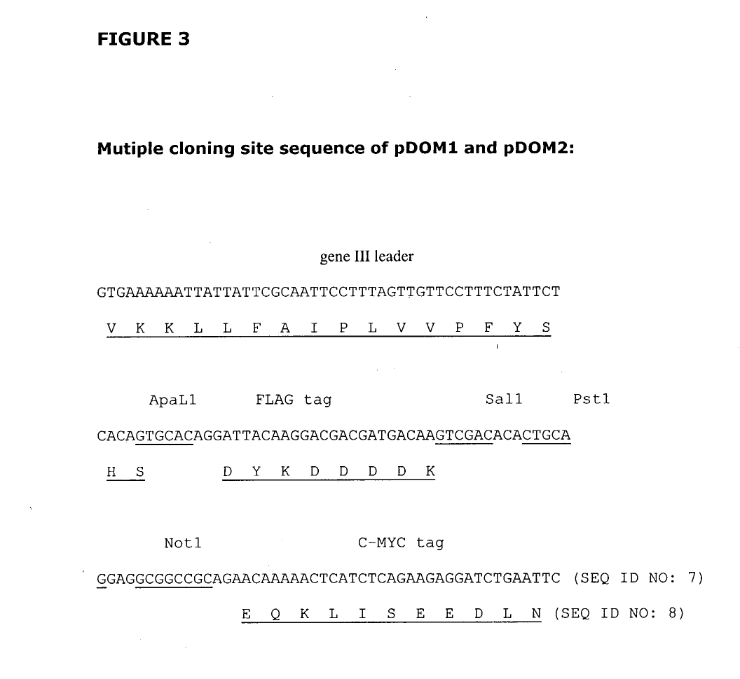

pDOM1 phage vector was constructed as follows: An oligocassette was prepared by annealing two oligonucleotides (DOMMRC1 and DOMMRC2, see Table 2). The cassette was ligated into Fd TET-DOG (McCafferty et al. Nature 1980, 348, 552) which was cut with ApaL1 and Not1. The ligated DNA was electroporated into electrocompetent E. coli TG1 cells. This creates the pDOM1 vector with a multiple cloning site as in FIGS. 1 and 3.

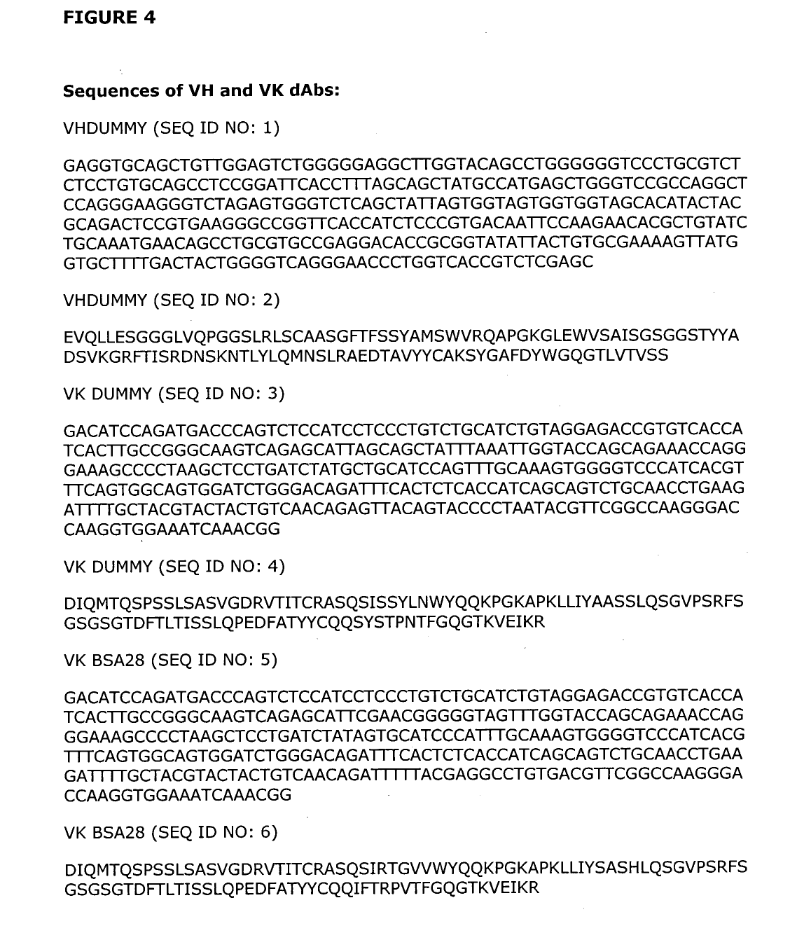

V_{H} and V_{K} domains were amplified with oligonucleotides (DOMMRC 9 and DOMMRC 10 for VH and DOMMRC 11 and DOMMRC 12 for VK) to append a SalI and NotI restriction sites. These PCR products were then cut with SalI and NotI and ligated into the Fd FLAG-myc phage vector cut with the same enzymes. Two VH dAbs i.e. VH dummy (a functional dAb with undefined specificity) and HEL4 (anti Hen-Egg-Lysozyme dAb) and two VK dAbs, i.e. VK dummy (a functional dAb with undefined specificity) and BSA28 (anti BSA dAb) were cloned into the pDOM1 phage vector. The sequences of these VH and VK dAbs are given in FIG. 4. The dummy sequences are germline variable domain sequences which do not have any target antigen binding capacity, but which can still bind generic ligands such as protein A and protein L. The dummy sequences are used as negative controls to test generic ligand binding in the absence of target antigen binding. The V_{H} dummy is used to assess protein A binding, and the V_{K} dummy is used to assess protein L binding.

TABLE 2
Sequences of oligonucleotides

| | | |
|---|---|---|
| DOMRC-1: | P-TGCACAGGATTACAAGGACGACGATGACAAGTCGACACACTGCAGGAGGC | (SEQ ID NO: 33) |
| DOMRC-2: | P-GGCCGCCTCCTGCAGTGTGTCGACTTGTCATCGTCGTCCTTGTAATCCTG | (SEQ ID NO: 34) |
| DOMRC-9: | GTGGTGTCGACAGAGGTGCAGCTGTTGGAGTCTGGGGGAG | (SEQ ID NO: 35) |
| DOMRC-10: | GAGTCAACTGCGGCCGCGCTCGAGACGGTGACCAGGGTTCCCTG | (SEQ ID NO: 36) |
| DOMRC-11: | GTGGTGTCGACAGACATCCAGATGACCCAGTCTCCATCCTC | (SEQ ID NO: 37) |
| DOMRC-12: | GAGTCAACTGCGGCCGCCCGTTTGATTTCCACCTTGGTCCCTTGG | (SEQ ID NO: 38) |
| DOM-6: | ATGGTTGTTGTCATTGTCGGCGCA | (SEQ ID NO: 39) |
| DOM-88: | CGCCAAGCTTTGGAGCCTTTTTTTTTGGAGATTTTTAACATGAAAAAATTA | (SEQ ID NO: 40) |
| | TTATTCGCAATTCC | |
| DOM-89: | GCGCGAATTCTTATTAATTCAGATCCTCTTCTGAGATGAG | (SEQ ID NO: 41) |
| DOM-57: | ATGAGGTTTTGCTAAACAACTTTC | (SEQ ID NO: 42) |

Construction of Expression Vector:

pDOM2 expression vector was constructed as follows: The multiple cloning site of pDOM1 was PCR amplified using oligonucleotides DOM-88 and DOM-89, (see Table 2).

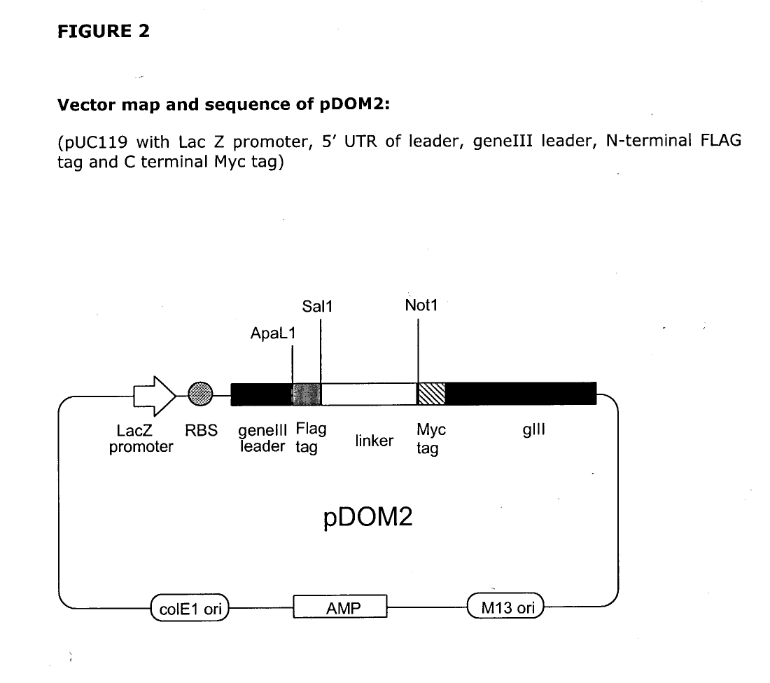

This PCR product was then cut with HindIII and EcoRI and ligated into the pUC119 vector (Sambrook et al, 1989) which was cut with the same enzymes. This creates the pDOM2 vector with a multiple cloning site as in FIGS. 2 and 3.

One VH dAb i.e. HEL4 was cloned into the pDOM2 vector. dAb HEL4 was PCR amplified from HEL4-pDOM1 using oligonucleotides DOM-6 and DOM-57, see Table 2).

This PCR product was then cut with SalI and NotI and ligated into pDOM2 vector which was cut with the same enzymes.

Protein A and Protein L ELISA with Monoclonal Phage.

VH and VK dummy phage particles were biotinylated using SULFO-NHS-BIOTIN (Perbio) to enable detection of the phage particles to Protein A or L. 96-well plates (Nunc, Maxisorp) were coated with Protein A (5 μg/ml in PBS) or protein L (1 μg/ml in PBS) overnight at 4° C. Plates were blocked with 2% skimmed milk powder/PBS (MPBS) for 2 hour at room-temperature (RT) and washed three times with PBS. Purified biotinylated phage in PBS (starting with 10e10 TU in the first well and then 4-fold serial dilutions) is mixed in 2% MPBS and incubated for 1 hour at RT on the coated plates. Plates are washed six times with PBS/0.05% Tween 20 (PBST). Binding phage were detected with streptavidin HRP conjugate, 1/2000 diluted in 2% MPBS. Plates are washed as above and developed as above.

Antigen ELISA with Monoclonal Phage:

96-well plates (Nunc, Maxisorp) were coated with BSA (10 μg/ml in PBS) or Hen-Egg-Lysozyme (5 μg/ml in PBS) or anti-FLAG antibody (Sigma, 1 μg/ml in PBS) overnight at 4° C. Plates were blocked with 2% skimmed milk powder/PBS (MPBS) for 2 hour at room-temperature (RT) and washed three times with PBS. Fifty μl of phage supernatant is mixed with an equal amount of 4% MPBS and incubated for 1 hour at RT on the coated plates. Plates are washed six times with PBS/0.05% Tween 20 (PBST). Antigen-binding phage were detected with anti-M13 HRP conjugate (Pharmacia), 1/5000 diluted in 2% MPBS. Plates were washed as above and ELISAs were developed using 3′3′5′5′-tetramethylbenzidine substrate in 0.1 M Na acetate, pH 6.0. The reactions were stopped with 1N HCl (50 μl per well) and the OD₄₅₀ was measured.

Antigen ELISA with Soluble dAbs:

96-well plates (Nunc, Maxisorp) were coated with Hen-Egg-Lysozyme (3 mg/ml in PBS), overnight at 4° C. Plates were blocked with 2% Tween/PBS (TPBS) for 1 hour at room-temperature (RT) and washed three times with PBS. Fifty μl of culture supernatant is mixed with an equal amount of 2% TPBS and incubated for 1 hour at RT on the coated plates. Plates are washed six times with PBS/0.05% Tween 20. Antigen-binding dAbs were detected with anti FLAG HRP (Sigma), protein A HRP (Pharmacia) or anti-myc mouse monoclonal (Sigma Cat No: M5546) followed by goat anti mouse IgG (Fc specific) HRP conjugate (Sigma Cat No: A0168). Plates were washed as above and ELISAs were developed using 3′3′5′5′-tetramethylbenzidine substrate in 0.1 M Na acetate, pH 6.0. The reactions were stopped with 1N HCl (45 ul per well) and the OD₄₅₀ was measured.

Design of Library

A phage library was constructed and selections were performed against 3 antigens. The library was created by introducing diversity in CDR1, CDR2 and CDR3 at positions that are highly diverse in the mature human repertoire. The diversified CDRs were randomly combined by cloning into a plasmid vector to create Library #1. This library was then PCR amplified and cloned in a newly constructed pDOM1 phage vector containing a geneIII leader sequence, N-terminal FLAG tag and C-terminal myc-tag to create Library #2 (VH) and Library #3 (VK). Selections were performed against three antigens: Antigen #1 (hen egg lysosyme) and human and mouse versions of another antigen (Antigens #2 and #3 respectively). Selections against Antigens #1 and #2 were successful.

Libraries are based on a single human framework for V_{H} (V3-23/DP-47 and J_{H}4b) and VK (O12/O2/DPK9 and JK1). The canonical structures (V_{H}: 1-3 and VK: 2-1-1) encoded by these frameworks are by far the most common in the human antibody repertoire. Side chain diversity is incorporated using diversified codons at positions in the antigen binding site that make contacts to antigen in known structures and are highly diverse in the mature repertoire.

Phage selections were performed as described previously (Griffiths et al. EMBO J 1994 13, p3245). Selections form the 3^{rd} round were re-cloned into expression vector pDOM2 as described above. Individual colonies were tested for binding to their antigen as described above.

Results

Phage Yield and Infectivity.

The phage yield is similar to that of dAb-phage without N-terminal tag (about 3×10e10 TU/ml of culture supernatant) and the infectivity is not affected.

Antigen Specific Phage Binding.

VH dummy phage particles bound to the anti-FLAG antibody and to protein A (no signal on protein L, nor to plastic). VK dummy phage particles bound to the anti-FLAG antibody and to protein L (no signal on protein A, nor to plastic). HEL4 phage particles bound to lysozyme (no signal on plastic). BSA28 phage particles bound to BSA (no signal on plastic).

Antigen Specific Soluble dAb Binding.

HEL4 soluble dAb binding to lysozyme could be detected with anti-FLAG and anti-myc antibody and also with protein A HRP (no signal on plastic).

Phage Library: Selection

Library selections were performed against Hen-Egg-Lysozyme, Antigen #2 and Antigen #3. Selections form the 3^{rd} round were re-cloned into expression vector pDOM2 as described above. Individual clones were tested as soluble dAbs for binding to their antigen.

Specific binders were selected against Hen-Egg-Lysozyme, and Antigen #2.

Conclusion

The results show that phage particles of VH or VK dAbs in the pDOM1 phage vector specifically bind antigen, protein A or protein L. In addition, the tag which is N-terminal of the dAb sequence can be detected. Phage yield or infectivity is not affected.

The results also demonstrate that dAb libraries cloned in the newly created pDOM1 phage vector are functional and that several antigen binding specificities can be isolated from this library. Some of the isolated dAb clones have a functional activity i.e. block binding of antigen to its ligand.

All of the above indicates that VH or VK dAbs are functional when expressed with an N-terminal tag and that antigen binding is not severely affected by tags.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A nucleic acid molecule comprising a first DNA sequence encoding the signal peptide of a bacteriophage protein linked at its 3′ end to a second DNA sequence encoding a tag wherein the second DNA sequence is linked at its 3′ end to a third DNA sequence encoding an immunoglobulin variable region polypeptide.

2. A polypeptide molecule comprising a first amino acid sequence comprising a signal peptide of a bacteriophage protein that is linked at it's C-terminus to the N-terminus of a second amino acid sequence comprising a tag, wherein the second amino acid sequence is linked at its C-terminus to a third amino acid sequence comprising an immunoglobulin variable region polypeptide.

3. The molecule of claim 1 or 2 wherein said bacteriophage protein is a bacteriophage coat protein.

4. The molecule of claim 1 or 2 wherein said tag binds to a protein ligand.

5. The molecule of claim 4 wherein said protein ligand is an antibody.

6. The molecule of claim 1 or 2 wherein said tag binds to a metal-chelate resin.

7. The molecule of claim 1 or 2 wherein said tag is selected from the group consisting of:
a fluorescent tag, a luminescent tag, or a chromogenic tag.

8. The molecule of claim 1 or 2 wherein said tag is selected from the group consisting of: Flag, His, Myc, HA, VSV and V5.

9. The molecule of claim 1 or 2 wherein said immunoglobulin variable region polypeptide comprises a light chain variable domain (V_{L}).

10. The molecule of claim 1 or 2 wherein said immunoglobulin variable region polypeptide comprises a heavy chain variable domain (V_{H}).

11. The molecule of claim 1 or 2 wherein said signal peptide is the signal peptide from a bacteriophage protein pIII or pVIII.

12. The molecule of claim 1 or 2 wherein said signal peptide is encoded by a sequence found in the genome of bacteriophage derived from a bacteriophage fd.

13. The nucleic acid molecule of claim 1 wherein said third DNA sequence is linked at its 3′ end to a bacteriophage coat protein.

14. The polypeptide molecule of claim 2 wherein said third amino acid sequence is fused at its C-terminus to a bacteriophage coat protein.

15. The molecule of claim 13 or 14 wherein said bacteriophage coat protein DNA sequence is found in the genome of a bacteriophage selected from the group consisting of: fl, fd, M13, and IKe.

16. A nucleic acid library comprising a plurality of nucleic acid molecules according to claim 1.

17. A polypeptide library comprising a plurality of polypeptide molecules according to claim 2.

18. A library of bacteriophage particles displaying on their surface a polypeptide molecule of claim 2.

19. A method for selecting from a repertoire of polypeptides a population of immunoglobulin variable region polypeptides that bind to a target ligand,
the method comprising contacting the polypeptide library of claim 17 with a target ligand and selecting a population of polypeptides which bind to the target ligand.

20. A method for selecting from a repertoire of polypeptides a population of immunoglobulin variable region polypeptides that bind to a target ligand, the method comprising
(a) expressing the library of claim 16 in a host cell to produce a polypeptide library; and
(b) contacting the polypeptide library with a target ligand and selecting a population of polypeptides which bind to the target ligand.

21. A composition comprising E. coli strain TB1, wherein said strain comprises the nucleic acid library of claim 16.

22. A composition comprising E. coli strain TB1, wherein said strain comprises the polypeptide library of claim 17.

23. A tagged polypeptide comprising an immunoglobulin variable region polypeptide, wherein said tagged polypeptide is produced by the cleavage of the signal peptide from the polypeptide of claim 2.

24. A nucleic acid vector comprising a DNA sequence encoding a signal peptide of a bacteriophage protein linked at its 3′ end to a second DNA sequence encoding a tag, which is in turn linked at its 3′ end to a third DNA sequence encoding an immunoglobulin variable region polypeptide.

25. The nucleic acid vector of claim 24, further comprising a lacZ promoter.

26. The nucleic acid vector of claim 24, further comprising a bacteriophage geneIII promoter.

27. The nucleic acid vector of claim 25, wherein said vector is pDOM1.

28. The nucleic acid vector of claim 26, wherein said vector is pDOM2.
